(19) United States
(12) Patent Application Publication
(10) Pub. No.: US 2024/0245819 A1
(43) Pub. Date: Jul. 25, 2024

(54) NITRIC OXIDE-RELEASING DISINFECTION INSERT

(71) Applicant: UNIVERSITY OF GEORGIA RESEARCH FOUNDATION, INC., Athens, GA (US)

(72) Inventors: Elizabeth BRISBOIS, Athens, GA (US); Manjyot Kaur CHUG, Athens, GA (US)

(21) Appl. No.: 18/561,613

(22) PCT Filed: May 19, 2022
(86) PCT No.: PCT/US2022/030045

§ 371 Date: Nov. 16, 2023

Related U.S. Application Data
(60) Provisional application No. 63/190,456, filed on May 19, 2021.

(51) Int. Cl. A61L 2/26; A61L 2/20; A61L 2/24; A61M 25/00

(52) U.S. Cl. CPC A61L 2/26; A61L 2/20; A61L 2/24; A61M 25/00; A61L 2202/11; A61L 2202/14; A61L 2202/24; A61M 2025/0019

(57) ABSTRACT

Disclosed herein are disinfection inserts comprising a fiber optic and a polymer surrounding at least a portion of the fiber optic (and methods of making and using the same). The polymer comprises a NO donor molecule that releases NO upon illumination of the polymer by the fiber optic. The disinfection inserts can be inserted into tubing, catheters, and/or extracorporeal devices and illuminated to release NO from the polymer. The released NO inactivates pathogens on or within the tubing, catheter, and/or extracorporeal device. The disinfection insert can be configured for removable attachment to the tubing, catheter, and/or extracorporeal device, such that it can be periodically replaced. Furthermore, the disinfection insert can be placed in optical communication with a controllable light source. The controllable light source can be coupled to a light source controller. The intensity and wavelength of the light can be varied to change the flux of NO.

### CROSS-REFERENCE TO RELATED APPLICATIONS

This application claims the benefit of and priority to U.S. Provisional Patent App. No. 63/190,456, filed May 19, 2021, which is incorporated herein by reference in its entirety.

### STATEMENT REGARDING FEDERALLY SPONSORED RESEARCH

This invention was made with U.S. Government Support under Grant No. R01 HL151473 awarded by the National Institutes of Health. The U.S. Government has certain rights in the invention.

### FIELD

This disclosure relates to disinfection devices, and more particularly to those for use in medical equipment.

### BACKGROUND

Catheters (e.g., intravascular (IV) catheters) are fundamental to the contemporary hospital practices and are frequently implanted in critically ill patients for the administration of drugs, fluids, blood transfusion, dietary solutions, and for hemodynamic monitoring. Approximately 90% of all the patients admitted to the hospital encounter some sort of intravenous therapy during their hospital stay. The typical duration of catheter use in clinical settings like emergency rooms, operating theaters, and intensive care units (ICUs) can range from minutes to months. While acute catheters may last up to one week, others, such as hemodialysis catheters, may be used from several months to years. Of all medical devices used in hospital settings, catheters have one of the highest rates of device-related infections. The infection causing bacteria can adhere on the catheter surface and colonize to develop biofilms. The primary contact of the bacterial cells on the surface of the catheter can emanate from the patient's own skin flora which can colonize the catheter lumen, triggering the bacteria to travel from the catheter insertion site into the vasculature. In the hospital-based setting, hematogenous seeding on the catheter from another contaminated site can be another possible source of infection, and occasionally the contamination of the catheter lumen occurs because the infusate itself is contaminated. These are predominant sources of morbidity and mortality in patients contributing to conditions like catheter related blood stream infections (CRBSIs) (e.g., bacteremia and sepsis).

Each year the occurrence of CRBSIs in the United States alone is estimated to be more than 250,000 incidences with a mortality rate of approximately 35%, with costs of approximately $2.3 billion. Catheter-related infections are increasing, especially due to the formation of biofilms. When a catheter becomes contaminated, the resultant solution is initiation of antibiotic lock therapy, and removal, and replacement of the catheter. In some critical cases, surgery may be required. Bacteria protected within biofilms require up to 1,000 times more dosage of antibiotics than their free floating (e.g., planktonic) counterparts. This heavy dosage can increase the possibility of antibiotic resistance across the bacterial species, engender economic burdens, and present a threat to native beneficial bacteria and other healthy organs of the body.

Therefore, there is an urgent need for the efficacious and harmless approaches that can not only handle the emergence but also the propagation of pathogenic microorganisms. To overcome this issue and enhance the bactericidal effect of medical devices, scientists have utilized different approaches that employ antimicrobial agents in the medical-grade polymers such as doping of silver in catheters, antiseptic solutions, peptides, and enzymes that can thwart the replication of bacteria or increase the susceptibility of antibiotics. Despite all these efforts, CRBSIs still remain one of the biggest concerns pertaining to biomedical devices.

### SUMMARY

Disclosed herein are disinfection inserts comprising a fiber optic and a polymer surrounding at least a portion of the fiber optic. The polymer comprises a NO donor molecule that is releasable upon illumination of the polymer by the fiber optic. The disinfection inserts can be inserted into tubing, catheters, and/or extracorporal devices and illuminated to release NO from the polymer. The released NO contacts and inactivates pathogens on or within the tubing or catheter. The disinfection insert can be configured for removable attachment to the tubing or catheter, such that it can be periodically replaced. Furthermore, the disinfection inserts, and specifically the fiber optic, can be placed in optical communication with a controllable light source. The intensity and wavelength of the light from the light source can be varied to change the flux of NO from the disinfection insert. The light source can be controlled by a light source controller that is wirelessly or electrically coupled to the light source.

Disinfection systems disclosed herein include a disinfection insert including a fiber optic and a polymer surrounding at least a portion of the fiber optic. The disinfection insert is configured to extend within a lumen of a medical tubing. The polymer includes a nitric oxide (NO) donor molecule. Some embodiments of the disinfection systems further include a light source in optical communication with the fiber optic of the disinfection insert. The disinfection insert can be illuminated by the light source. In the illuminated state, the disinfection insert releases NO into the catheter lumen. In some implementations, the catheter is an indwelling catheter. Some embodiments further include a light source controller.

In some embodiments, the disinfection insert further includes a fastener configured to removably attaching the disinfection insert to a medical tubing. The fiber optic can extend the length of the fastener. In some embodiments, the fastener can include a flush port. In some embodiments, the light source includes a coupling for attachment to the fiber optic. Certain implementations may enable the light source to be removably attached to the disinfection insert. In some embodiments, the fiber optic is a side glow fiber optic.

In some embodiments, the polymer is silicone rubber. The polymer can be, for example, a siloxane-based polyurethane elastomer or a thermoplastic silicone-polycarbonate urethane. The polymer can be coated directly onto the fiber optic, or the polymer can be a tube defining a space between an interior surface of the tube and the fiber optic.

In some embodiments, the NO donor molecule is an S-nitrosothiol (RSNO). In some embodiments, the disinfection insert releases NO at a flux between 0.1×10⁻¹⁰ mol cm⁻² min⁻¹ and 100×10⁻¹⁰ mol cm⁻² min⁻¹.

In some embodiments, the light source delivers light of wavelengths ranging from 200 nanometers to 700 nanometers, and/or of variable intensity. The light source can include a battery, in some implementations. The light source controller can be configured to control the wavelength and/or intensity of light from the light source. In some implementations, the light source controller is coupled to the light source via wireless communication. In some implementations the light source controller is electrically coupled to the light source.

Methods of making a disinfection insert includes incorporating (e.g., impregnating) the NO donor molecule into the polymer and coupling the fiber optic to the polymer. In some embodiments, coupling the fiber optic to the polymer includes dipping a portion of the fiber optic into a liquid form of the polymer, such that the polymer coats at least a portion of the fiber optic. In some embodiments, coupling the fiber optic to the polymer comprises attaching a solid form of the polymer to the fiber optic such that the polymer surrounds at least a portion of the fiber optic.

Methods of making the disinfection insert can include coupling a fastener to the disinfection insert, the fastener being removably attachable to a tubing, catheter, and/or extracorporeal device. The methods further include placing the fiber optic into optical communication with a light source, for example, by attaching the fiber optic to a coupling on the light source.

In the methods of making the disinfection insert, the step of incorporating an NO donor molecule into the polymer can include incorporating an RSNO into the polymer (for example, by soaking a solid form of the polymer in a solution comprising an RSNO, by mixing an RSNO into a liquid form of the polymer, or by covalently bonding an RSNO to the polymer backbone). The methods can include incorporating combinations of NO donor molecules, or combinations of RSNOs, into the polymer.

Methods of disinfecting a tubing include steps of inserting an elongated disinfection insert into a lumen of the tubing, illuminating the disinfection insert, releasing NO from a polymer of the disinfection insert, contacting pathogens on or within the tubing with the NO from the polymer, and inactivating at least a portion of the pathogens on or within the tubing via contact with the NO. The NO can inactivate pathogens within both the lumen of the tubing and within the walls of the tubing (by diffusion). In some embodiments, the tubing can be a part of a medical catheter. In some embodiments, the tubing can be part of an extracorporeal medical device. In some embodiments, the extracorporeal medical device is one of an endotracheal tube, a wound dressing or wound patch, a photodynamic therapy device, a cardiopulmonary bypass device, a hemodialysis device, a medical port, a feeding tube, or an intestinal tube.

Some example methods include a step of fastening the disinfection insert to an end of the tubing. The disinfection insert may be replaceable in some examples, such that the method further comprises unfastening the first disinfection insert from the end of the tubing and replacing it by fastening a second disinfection insert to the end of the tubing. Some example methods of disinfecting a tubing can include attaching the disinfection insert to a coupling on a light source.

The methods of disinfecting a tubing can further include a step of activating a light source that is in optical communication with the disinfection insert. In some embodiments, the disinfection insert includes a fiber optic, and illuminating the disinfection insert comprises illuminating the fiber optic. Illuminating the fiber optic causes illumination of an NO donor within the polymer. In some embodiments, the polymer includes a RSNO, and the step of releasing NO from a polymer comprises releasing NO from the RSNO.

Some methods of disinfecting the tubing further include a step of varying a flux of NO from the polymer by changing an intensity of light from the light source, and/or a wavelength of light from the light source. The NO from the polymer can be released at a flux between 0.1×10⁻¹⁰ mol cm⁻² min⁻¹ and 100×10⁻¹⁰ mol cm⁻² min⁻¹. In some configurations, the light source is controlled by a light source controller. In some configurations, the light source is controlled wirelessly.

### BRIEF DESCRIPTION OF DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements. The drawings are not necessarily drawn to scale.

FIG. 1A shows an in-use schematic of a catheter disinfection insert releasing nitric oxide (NO) from a polymer and into a catheter lumen when a side-glow fiber optic of the catheter disinfection insert is illuminated by a light source, according to some embodiments.

FIG. 1B shows a front side-view of the catheter disinfection insert and light source of FIG. 1A with the polymer of the catheter disinfection insert spaced from the coupling between the fiber optic and the light source, according to some embodiments.

FIG. 1C shows a schematic of the catheter disinfection insert of FIG. 1A when inserted into an intravenous (IV) catheter, according to some embodiments.

FIG. 1D shows a back side-view of the light source and catheter disinfection insert of FIG. 1A, according to some embodiments.

FIG. 1E shows an end view of the catheter disinfection insert and light source of FIG. 1D, according to some embodiments.

FIG. 1F shows a transverse cross section of the catheter disinfection insert of FIG. 1A, according to some embodiments.

FIG. 1G shows an alternate end view of the catheter disinfection insert and light source of FIG. 1D, according to some embodiments.

FIG. 1H shows a longitudinal cross section of the catheter disinfection insert of FIG. 1A, according to some embodiments.

FIG. 1I shows a coupling of the light source of FIG. 1A engaging with a fastener to connect the catheter disinfection insert to a catheter, according to some embodiments.

FIG. 1J shows the light source and fastener fully coupled to the catheter, according to some embodiments.

FIG. 2 is a graph showing a quantification of S-nitroso-N-acetylpenicillamine (SNAP) impregnation in silicone rubber (SR) samples, according to some embodiments.

FIG. 3 is a graph comparing the NO release from a SNAP impregnated sample with and without application of a light source, according to some embodiments.

FIGS. 4A-4D are graphs showing a verification of wavelength of light emitted by the catheter disinfection insert of FIGS. 1A-1J when connected to an LED light source, according to some embodiments.

FIG. 5 is a graph showing a comparison of NO release from the catheter disinfection insert of FIGS. 1A-1J at physiological temperature (e.g., 37° C.) in the dark and photoinitiated at 100% light intensity various colors of light, according to some embodiments.

FIGS. 6A and 6B are graphs showing an example real-time modulation of NO release from the catheter disinfection insert of FIGS. 1A-1J, according to some embodiments.

FIG. 7 is a graph showing the amount of SNAP in a phosphate buffer saline (PBS) soaking buffer the catheter disinfection insert of FIGS. 1A-1J is incubated at 37° C. in the dark (SNAP) and 100% white light intensity (SNAP-light), according to some embodiments.

FIG. 8A is a first example graph showing the antibacterial activity of the catheter disinfection insert after a 4-hour exposure to S. aureus, calculated as the log of the colony forming units (CFU) cm⁻² of polymer surface area, according to some embodiments.

FIG. 8B is a second example graph showing the antibacterial activity of the catheter disinfection insert after a 2-hour exposure to S. aureus, according to some embodiments.

FIG. 8C is a second example graph showing the antibacterial activity of the catheter disinfection insert after a 4-hour exposure to E. coli, according to some embodiments.

FIG. 8D is a graph showing the antibacterial activity of the catheter disinfection insert after a 4-hour exposure to S. aureus as calculated as the log of the colony forming units (CFU) cm⁻² of polymer surface area, according to some embodiments.

FIG. 8E shows an example agar plate with viable S. aureus after a 4-hour exposure to a control insert and the catheter disinfection insert of FIG. 1A, according to some embodiments.

FIG. 9 is a graph showing the cytocompatibility of the catheter disinfection insert evaluated against NIH 3T3 mouse fibroblast cell line in a 24 hour cell viability assay using a CCK-8 cell viability kit, according to some embodiments.

FIG. 10 is a graph demonstrating the impact of UV and EO sterilization on SNAP impregnated SR, according to some embodiments.

FIG. 11 is a graph illustrating the tunable release of NO by the catheter disinfection insert of FIGS. 1A-1J by modulating the light source, according to some embodiments.

FIG. 12A is a diagram showing an example of the catheter disinfection insert of FIGS. 1A-1J in operation, according to some embodiments.

FIG. 12B shows another example diagram of the catheter disinfection insert of FIGS. 1A-1J in operation, according to some embodiments.

FIG. 13 shows the chemical structure of the NO donor SNAP, according to some embodiments.

### DETAILED DESCRIPTION

Nitric oxide (NO) is an innate signaling diatomic molecule utilized by the body's defense systems for fighting infection-causing microorganisms, preventing platelet activation, reducing localized and chronic inflammations, and enhancing wound healing. Endogenous synthesis of NO in the body occurs via nitric oxide synthase (NOS) enzymes which convert the amino acid L-arginine into citrulline and NO. Considering the potential benefits of endogenous NO, various studies have been designed that can utilize these benefits synthetically by either incorporating/impregnating the NO donors in the polymer matrix that will release their NO payload or using a generation mechanism to stimulate the release of endogenous NO in blood. Nitric oxide donors like S-nitrosothiols (RSNO) incorporated into a polymer substrate can mimic endogenous NO release levels, such as endothelial cells that release NO at a surface flux of 0.5−4×10⁻¹⁰ mol cm² min⁻¹ to prevent platelet activation and adhesion. Macrophages and neutrophils utilize NO synthesized via the inducible nitric oxide synthase (iNOS)>1 microMolar NO which demonstrates antimicrobial activity by promoting biofilm dispersal and preventing the adherence of planktonic bacteria.

NO can be loaded into polymeric substrates and released in a tunable, controlled manner using a variety of triggering mechanisms. To enhance the NO payload and extend lifetime of NO release, several distinct frameworks have been developed as NO-releasing or NO-generating mechanisms at the polymer interface. Such engineered polymer surfaces that can either release or generate NO have included: polymers with physically dispersed NO donors, polymers with NO donors covalently bound to the polymer backbone, and polymers that include metal catalysts that generate NO from endogenous RSNO species. The RSNO donors like S-nitroso-N-acetylpenicillamine (SNAP) and S-nitrosoglutathione (GSNO) have been recognized to have extended storage capacity in crystallized form and can emit NO either photochemically, thermally by heat, light or metal ions (Cu²⁺, Se, Zn etc.). Strategies to modulate the levels of NO by dip coating the NO-releasing matrix with a hydrophobic polymer layer such as CarboSil, silicone, E2As and PVC have been developed to prevent leaching of NO donor. Although these approaches demonstrate advantageous biocompatibility properties, eventually the NO reservoir will be depleted which limits the functional lifetime for long-term catheter applications. It has been reported that RSNOs and RSNO-based polymers can photocatalytically release their NO payload. The characteristic absorption maxima for the RSNOs occurs at wavelengths 340 nm and 520-590 nm with corresponding to n→π* electronic transition of S—NO functional group that have been primarily associated with their decomposition.

Another approach of addressing medical device infections with light has been with the use of UVC or photodynamic therapies. It has been well established that the blue light holds great potential with respect to killing infection causing microorganisms. Staphylococcus aureus is the predominant bacterial strain associated with CRBSI arising from IV catheters, with a mortality rate ranging from 20%-30%. The susceptibilities of S. aureus and E. coli to photodynamic inactivation by visible light have been previously reported. Furthermore, UV irradiation from LEDs in polymer tubes can be used to destroy bacteria and other microbes. However, direct UVC exposure can cause undesirable effects ranging from inflammation and premature aging to serious burns and cancer.

### Overview

Referring generally to the figures, a catheter disinfection insert is shown that overcomes the aforementioned limitations of NO and light-mediated microbe killing. The catheter disinfection insert, also generally referred to as “the insert,” includes a side glow fiber optic surrounded by a NO-releasing polymer, creating a device that can be inserted into a medical catheter, or any type of tubing for that matter, to prevent and eradicate viable pathogens. NO has broad-spectrum disinfection activity against bacteria, virus, fungus, and parasites. In some examples, the insert can be left in an indwelling catheter in order to treat and prevent infections that might otherwise occur on catheter surfaces. For example, when the catheter is not in use by clinicians for blood draws or infusions, it could be filled with a saline lock solution and the insert. The insert could be illuminated, constantly or periodically, in order to kill pathogens on the interior surfaces of the catheter. Furthermore, the device could be utilized to disinfect other luminal medical devices like endotracheal tubes, wound dressing bandages, access ports, dialysis or cardiopulmonary bypass machines, for example.

Certain indwelling catheters have interior surfaces coated with antimicrobial agents. The disinfection insert disclosed herein is an improvement because, unlike coated catheters, the insert can be removed from the catheter and replaced periodically as needed upon depletion of the NO from the polymer. This negates the need to replace the entire catheter. Advantageously, the insert can also be disposable. Accordingly, the insert may also be referred to herein as a disposable catheter disinfection insert (DCDI).

### Disinfection Insert for Catheters

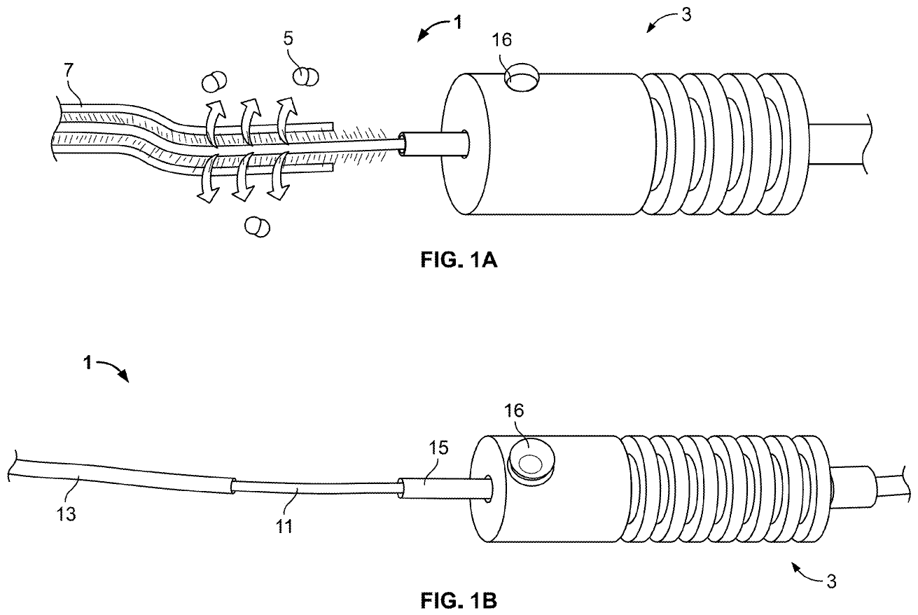

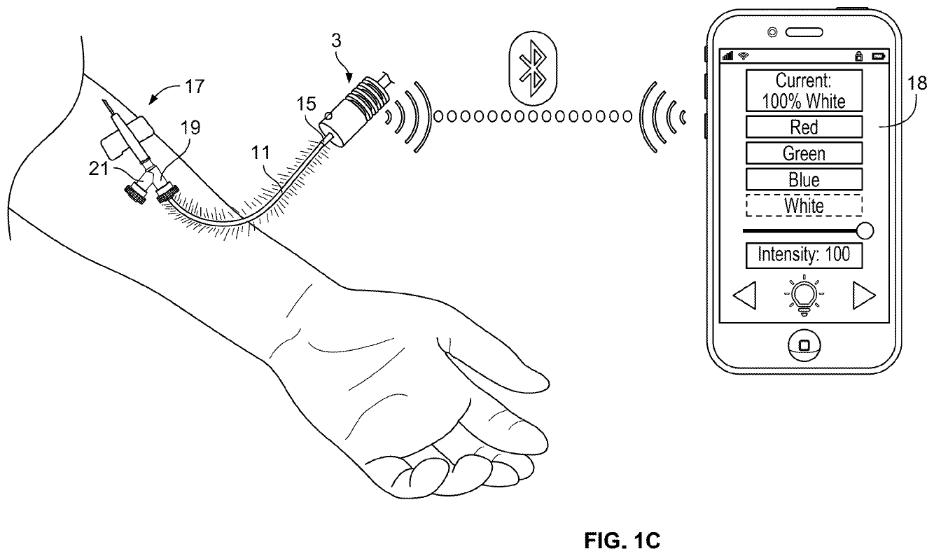

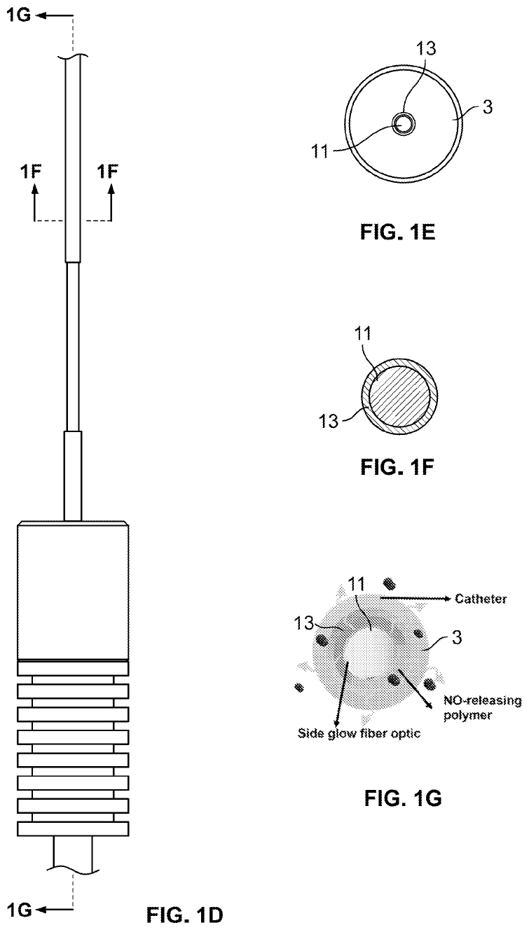

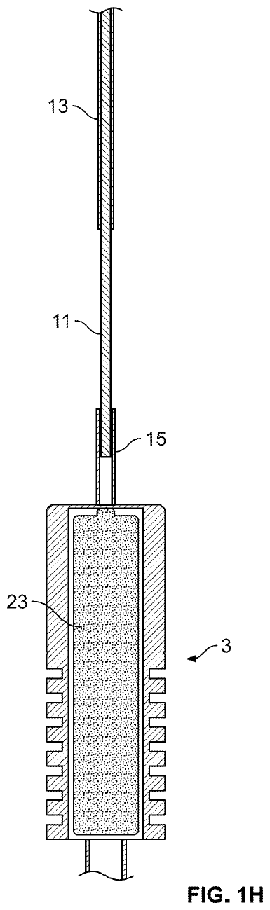

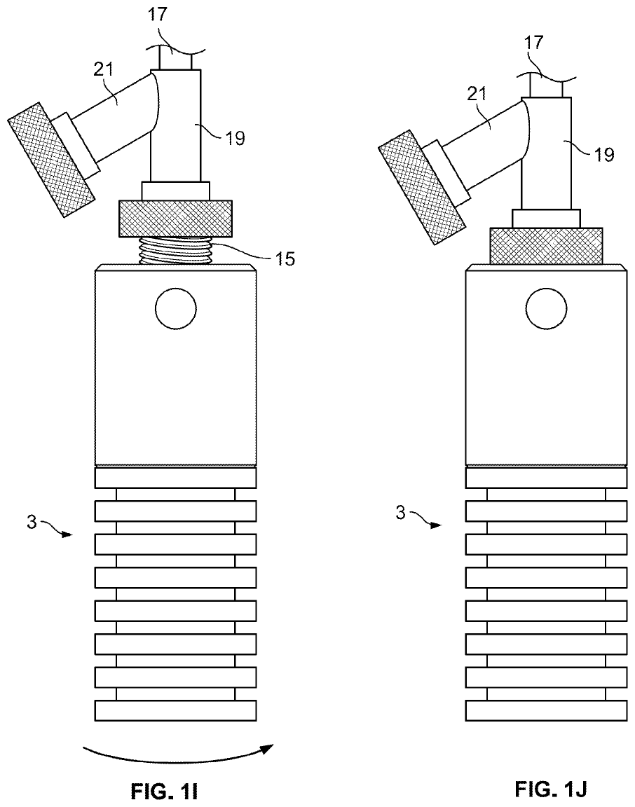

Turning first to FIGS. 1A and 1i, a catheter disinfection insert 1 is shown. As mentioned above, the catheter disinfection insert 1, or simply “insert 1,” is generally configured to disinfect a medical catheter, shown as catheter 7. As shown, insert 1 may be in optical communication with a light source 3, described in greater detail below. In some embodiments, insert 1 is illuminated by light source 3 which causes the release of nitric oxide (NO) 5 into a lumen 9 of catheter 7. It should be noted that the attachment of insert 1 to the catheter 7 is not shown in FIG. 1A but will be discussed in greater detail below. During use, insert 1 is positioned extending into lumen 9 of catheter 7, as illustrated in FIG. 1A. As shown in FIG. 1B, insert 1 can include a fiber optic 11 surrounded by a polymer 13. Specifically, the example diagram of FIG. 1B shows insert 1 with polymer 13 slightly spaced from light source 3, thereby exposing a length of fiber optic 11. However, it should be appreciated that polymer 13 may be positioned directly adjacent to light source 3. In some embodiments, the amount and positioning of polymer 13 can be varied, so long as polymer 13 is positioned inside lumen 9 of catheter 7 during use.

Referring now to FIG. 1C a diagram of insert 1 being used in a medical setting is shown. Specifically, insert 1 is shown to be partially inserted into an IV catheter 17, which is position in the arm of a patient. Though the example shown is an IV catheter, other types of catheters and/or tubing and/or cannulas could benefit from use of insert 1 (for example, urinary catheters, insulin cannulas, wound healing devices, peritoneal dialysis catheters, hemodialysis catheters). Indwelling catheters—those designed to stay inserted for longer procedures or treatments—can especially benefit from use of insert 1 because pathogens are given ample time to colonize inner surfaces of indwelling catheters (note, however, that NO can diffuse through walls of catheters and tubing to disinfect inner surfaces, outer surfaces, and any pores extending between the inner and outer surfaces).

Insert 1 may be removably attached to the proximal end of the catheter 17 by a fastener 19, in some examples. The fastener 19 advantageously enables insert 1 to be replaced. In the aspect of FIG. 1C, fiber optic 11 of insert 1 is inserted through the length of fastener 19 and into the IV catheter 17. In some embodiments, fastener 19 can be structured as a Y-connector, including a flush port 21, as shown in FIG. 1C. Flush port 21 allows for the injection of saline into the catheter without removing insert 1. Flushing intravascular catheters with saline can be used to instill fresh lock solutions and/or help maintain patency so that a blood clot does not form at the distal tip of the catheter.

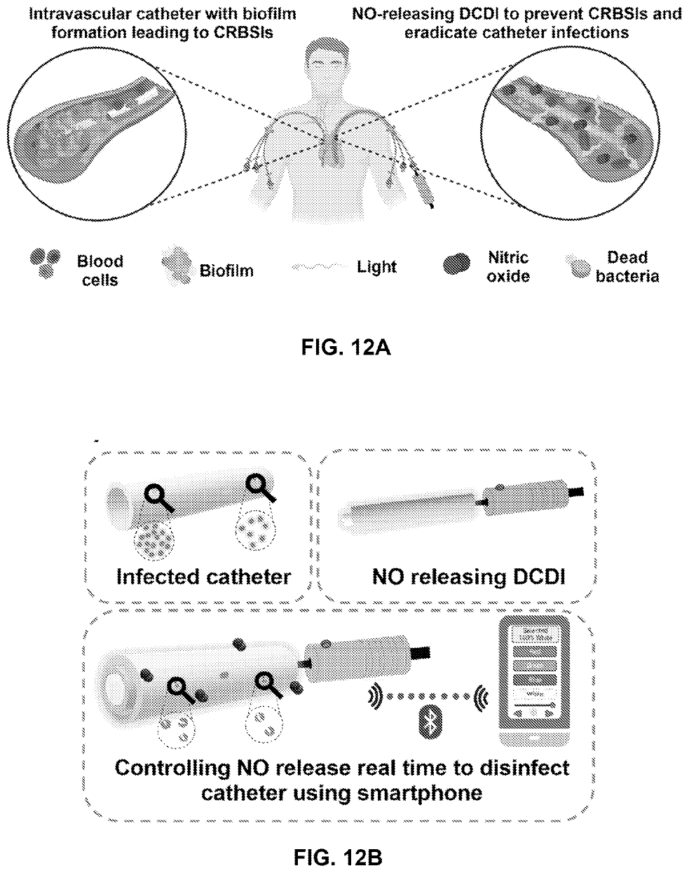

Turning briefly to FIGS. 12A and 12B, additional diagrams of insert 1 being used in a medical setting are shown. Specifically, on the left-hand side of FIG. 12A, an example IV catheter is shown with biofilm formation, which can lead to catheter-related bloodstream infections (CRBSIs). In contrast, the right-hand side of FIG. 12A shows an example IV catheter that is treated with insert 1. As shown, insert 1 drastically reduces the potential for catheter infections and CRBSIs by killing or deactivating bacteria within and around the catheter, thereby extending the usage lifetime of medical devices and drastically reducing associated treatment cost. In FIG. 12B, insert 1 is shown to be inserted into an infected catheter and illuminated. In some embodiments, as mentioned above, light source controller 18 wirelessly controls light source 3 to cause the release or emission of NO molecules from insert 1, as shown.

FIGS. 1I and 1J show closer views of an example connection between light source 3 and the catheter 17. In this aspect, the coupling 15 extending from light source 3 is a Luer lock that can be screwed into fastener 19, as shown in FIG. 1I. Fiber optic 11 of insert 1 is hidden from view, but extends through coupling 15, fastener 19, and into catheter 17. FIG. 1J shows light source 3 fully coupled to fastener 19, which is coupled at its other end to catheter 17. Note that this particular configuration is intended to be an example. Other configurations for joining light source 3 to the catheter 17 could use alternative fastening means (snap fit or press fit couplings, for example). In other configurations, the light source 3 might be distanced from the proximal end of the fastener 19, such that insert 1 extends a distance between light source 3 and the catheter 17. Alternatively, a single component might serve as both the fastener 19 and the light source coupling 15, continuously connecting light source 3 to the catheter 17 with insert 1 extending through the fastener/coupling component.

Light source 3 is in optical communication with fiber optic 11. In the aspect of FIG. 1i, coupling 15 serves as the attachment between light source 3 and fiber optic 11. Coupling 15 is depicted as a tubing bonded to fiber optic 11. However, the coupling 15 may take other forms. For example, fiber optic 11 can be held in place with one or more screws, clips, ferrules, couplers or adhesives. Adhesives permanently connect the NO coated fiber optic to light source 3. However, in some embodiments, coupling 15 enables removable attachment of light source 3 to insert 1. Screws, clips, ferrules, couplers, etc. allow for light source 3 to be re-used-insert 1 that is attached to one of these connectors can be replaced as needed. In some embodiments, light source 3 is configured to deliver light of wavelengths ranging from 200 nm to 700 nm, and of variable intensity. In some embodiments, light source 3 emits light in a smaller range of wavelengths, between 450 and 650 nm. Screw 16 on the external surface of light source 3 extends into light source 3 and tightly secures fiber optic 11 to light source 3. In alternative examples, screw 16 could be replaced with other types of fasteners, clips or adhesives to bind fiber optic 11 tightly to light source 3.

In some embodiments, light source controller 18 is configured to control the wavelength of light emitted from light source 3, the intensity of light emitted from light source 3, or both. In some embodiments, light source controller 18 can be used to program the duration of time light source 3 will be activated, or to otherwise set temporal programs that vary the wavelength and/or intensity of light emitted from light source 3 in a pre-determined pattern. In the aspect shown in FIG. 1C, light source 3 is in wireless communication with a light source controller 18. In some embodiments, light source 3 and light source controller 18 wirelessly communicate via a suitable short-range wireless protocol, such as Bluetooth®. Accordingly, while not explicitly shown in the figures, each of light source 3 and light source controller 18 may include a short-range wireless transceiver, such as a Bluetooth® transceiver or a WiFi® transceiver. In other embodiments, light source 3 and light source controller 18 communicate wirelessly via a network (e.g., the Internet, a VPN, etc.) or by another type of wireless communication network (e.g., a cellular network).

In some embodiments, light source controller 18 implements a mobile phone application. In some such, light source controller 18 may include at least one processor and a memory that can store instructions (e.g., software) for execution by the at least one processor. Because the at least one processor and memory are internal to light source controller 18, they are not explicitly shown in FIG. 1C. In some embodiments, memory stores a software application (e.g., a “smartphone application”) that can be executed by the at least one processor to generate the interface shown in FIG. 1C and to cause light source controller 18 to perform various operations described herein. However, the disclosure is not limited by any particular light source, user interface, or light source controlling technology. In other aspects, light source controller 18 may be connected by wireless means other than Bluetooth® technology, or it may be physically connected by electrical wiring, for example. Likewise, in other aspects, light source controller 18 may be a computer application or be a manual switch. In some embodiments, light source controller 18 may be positioned on, or part of, light source 3.

Fiber optic 11 is a side emitting, or side glow, fiber optic, with cladding that enables partial escape of the light along the length of fiber optic 11. FIG. 1D shows another side view of insert 1 and light source 3, rotated 180 degrees around the axis as compared to the view from FIG. 1B. FIG. 1E is an end view looking at the distal end of insert 1 and light source. FIG. 1F is a cross section taken perpendicular to the longitudinal axis were indicated on FIG. 1D. Additionally, FIG. 1G shows the same cross section as FIG. 1F, along with an illustration of NO molecules being emitted from polymer 13 due to illumination of fiber optic 11.

As shown in FIGS. 1F and 1J, polymer 13 may be coated directly onto fiber optic 11. However, in another aspect, polymer 13 is a pre-formed tube into which fiber optic 11 is inserted. In this aspect, a space may exist between an interior surface of polymer 13 and fiber optic 11. Insert 1 is not limited to any particular diameter. The insert diameter can be modified to best suit the particular catheter or tubing that insert 1 is designed to disinfect. Likewise, the amount of polymer 13 can vary; the thickness of polymer 13 (measured radially from the exterior surface of fiber optic 11) can be thicker or thinner, depending upon the use for which it is intended. For example, the ratio of polymer 13 to fiber optic 11 may be greatest for catheters that will be used for the longest durations.

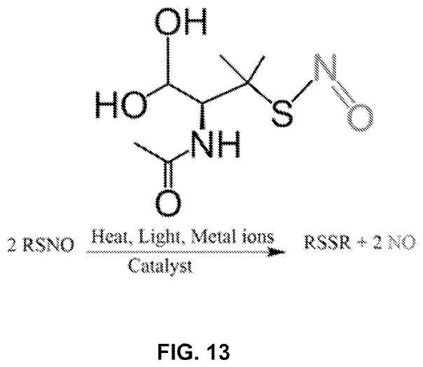

Polymer 13 is loaded with a NO donor molecule to release nitric oxide. In some embodiments, as shown in FIG. 1A, the NO donor molecule is photosensitive and enables light-initiated release of nitric oxide 5 upon illumination of the underlying fiber optic 11. In some examples, the NO donor molecule is an S-nitrosothiol (RSNO). Some examples of discrete RSNOs include, but are not limited to S-nitrosoglutathione (GSNO), S-nitroso-N-acetylpenicillamine (SNAP), S-nitrosocysteine (CysNO), etc., and derivatized discrete RSNOs. Derivatized RSNOs may be modified with alkyl group(s). Turning briefly to FIG. 13, a chemical structure of a NO donor SNAP molecule is shown. As described herein, RSNOs like SNAP can be triggered by the stimulus of heat, light, or metal ions to cleave the S—N bond and release NO.

As examples, a derivative may have an alkyl group attached to the free carboxyl group of SNAP and/or may have a longer alkyl (i.e., longer than acetyl) attached to the amine group of S-nitrosopenicillamine. As an example, an ester linkage may be formed between the desired alkyl group and the free carboxyl group of SNAP. As another example, a long chain alkyl (including from 4 to 10 carbon atoms) may replace the acetyl group of SNAP so that the long chain alkyl is attached to the amine nitrogen. As other examples, a sugar may be attached to the carboxyl group of SNAP (e.g., glucose-SNAP, mannose-SNAP, fructose-SNAP, etc.).

Generally, the disclosure is not limited to a specific type of polymer 13 for loading with the NO donor molecule. Various types of polymers may be suitable. For example, polymer 13 can be a silicone rubber, a siloxane-based polyurethane elastomer, a thermoplastic silicone-polycarbonate urethane, or a mixture thereof. These and other examples of NO loaded polymers are described in U.S. Pat. No. 9,566,372 and U.S. Patent Application Publication No. 2015/0366831, each of which is disclosed herein by reference in its entirety. The flux of NO from the surface of polymer 13 is preferably between 0.1×10-10 mol cm⁻² min⁻¹ and 100×10⁻¹⁰ mol cm⁻² min⁻¹.

FIG. 1H is a cross section taken parallel to the longitudinal axis were indicated on FIG. 1D. In the aspect shown in FIG. 1H, light source 3 is powered by a battery 23. Battery 23 may represent a single battery or multiple batteries. For example, battery 23 may be formed up multiple battery cells. In various embodiments, battery 23 can be disposable or rechargeable. In embodiments where battery 23 is disposable, battery 23 may also be removable from light source 3. Battery charging technology can include, for example, hard-wire charging, inductive charging, or solar charging. Accordingly, in some embodiments, light source 3 may include a charging port (not shown), such as a micro-USB or USB-C port, for charging battery 23. However, in other aspects, light source 3 could be connected directly to a power source.

In some embodiments, although not explicitly shown in the figures, light source 3 may also include a button or other user interface (e.g., a switch) for turning the device on/off. For example, light source 3 may include a button that, when pressed a first time (e.g., by a user), causes light to be emitted and, when pressed a second time, turns off the emission of light. In some embodiments, light source 3 may include a button that switches light source 3 between different operating modes. For example, the button may allow a user to select an intensity or color of light emitted. In some embodiments, light source 3 includes indicator LEDs or another type of user interface for indicating that light source 3 is turned on/off, and/or for indicating an intensity of the emitted light in embodiments where light source 3 is operable at varying intensities (e.g., brightness levels). For example, light source 3 may include at least four LEDs indicating intensities of 25%, 50%, 75%, and 100%; although, it should be appreciated that any type of indicator and any interval can be used (e.g., a digital display showing 0%-100%). In some embodiments, light source 3 may include a user interface (e.g., multiple LED indicators) for indicating a charge level of battery 23.

### Methods of Making a Disposable Catheter Disinfection Insert (DCDI)

Methods of making insert 1, also referred to herein as a DCDI, include incorporating, loading, swelling, doping, or impregnating a NO-donor molecule into a polymer 13. In some embodiments, making insert 1 includes conjugating or immobilizing the NO-donor moiety to polymer 13. The NO-donor loaded polymer 13 is then coupled to fiber optic 11 to polymer 13. In some embodiments, however, polymer 13 is coupled to fiber optic 11 before it is loaded with the NO donor molecule. In other embodiments, polymer 13 can first be impregnated with precursor molecules and later nitrosated to form an NO-rich molecule. In some embodiments, a fastener is coupled to insert 1 for removable attachment to a catheter 7. In some embodiments, fiber optic 11 is placed into optical communication with a light source 3, for example, by attaching fiber optic 11 to a coupling 15 on light source 3.

In some exemplary methods, the NO donor molecule is mixed into a liquid form of polymer 13, and fiber optic 11 is dipped into the liquid polymer to dip coat fiber optic 11, or the liquid polymer 13 is otherwise applied to the surface of fiber optic 11. Polymer 13 may coat any desired surface area of fiber optic 11.

In some embodiments, a solid form of polymer 13 may be loaded with the NO donor molecule, for example, by soaking a solid form of polymer 13 in a solution comprising the NO donor molecule. The method of making insert 1 then further includes coupling the solid polymer 13, loaded with the NO-donor molecule, to fiber optic 11. In some embodiments, adhesives or other bonding mechanisms may be used to couple the solid form of polymer 13 to fiber optic 11. Another alternative might be to apply adhesive glue on fiber optic 11 and then place polymer 13 on the fiber (so the adhesive is in between the fiber and polymer 13, holding everything together). Again, any desired surface area of fiber optic 11 may be covered with polymer 13 in this manner.

In some exemplary methods, the NO donor molecule is conjugated to a photosensitizer molecule that is mixed in with the polymer. For example, the NO donor molecule can be covalently immobilized to a titanium dioxide (TiO₂) particle. TiO2 particles can exhibit antibacterial properties once they are irradiated with light. Conjugating the NO donor molecule to a photosensitizer molecule such as TiO₂ can synergistically increase the antibacterial properties of the insert due to dual action of NO and the photosensitizer molecule.

Suitable NO adducts (examples of which include discrete adducts) are generally those exhibiting capability of embedding (either by covalent attachment and/or dispersion) into the polymer matrix and exhibiting process preparation stability.

In some exemplary methods, the NO donor molecule can be covalently bound to a backbone of the polymer 13. Since SNAP is a small molecule which has the tendency to leach out from a polymer matrix (e.g., polymer 13) over time, an alternative method of conjugating polymer 13 to SNAP and coating fiber optic 11 is also described herein. Specifically, in some embodiments, hydroxy-terminated polydimethylsiloxane (PDMS-OH, 2550-3750 cst, 800 mg) is dissolved in anhydrous toluene (5 mL), which is then supplemented with (3-aminopropyl) trimethoxysilane (APTMS, 150 mg) and dibutyltin dilaurate (DBTDL, 3.4 μL). The solution is left stirred overnight. N-acetyl-D,L-penicillamine thiolactone (NAPTH, 150 mg) is then added to the reaction vessel which is stirred for an additional 24 hours. Afterwards, the reaction mixture can be nitrosated using t-butyl nitrite (1.2 mL) that was first purified with Cyclam to remove copper stabilizing agents. This final nitrosated solution can then be used for subsequent dip-coating processes, as described herein. Alternatively, in some embodiments, the final nitrosated solution can be used to produce an NO-releasing polymer (e.g., polymer 13) which is then attached (e.g., glued) to fiber optic 11.

In some embodiments, the alternative fabrication method described herein includes coating approximately 3 cm of a 10 cm fiber optic (e.g., fiber optic 11) by dipping the fiber optic in the above-discussed SNAP-PDMS solution five times, with a one minute interval between each topcoat. The coated samples are then allowed to air dry overnight at room temperature and an additional 24 hours in a vacuum desiccator (e.g., to ensure all the solvent is evaporated from the samples). The coated fiber optics are then connected to light source 3 and operated in a “continuous light mode”. In other words, light source 3 can be used to trigger the NO release from insert 1.

### Methods of Disinfecting a Tubing

With the basic structure insert 1 and medical catheter system being thusly disclosed, a greater appreciation of the construction and benefits may be gained from the following discussion of the operation. It is to be noted that this discussion is provided for illustrative purposes only.

Disclosed herein are methods of disinfecting a tubing. Generally, the methods include inserting an elongated disinfection insert into a lumen of a tubing or medical catheter, illuminating insert 1, releasing NO from a polymer of insert 1, and contacting pathogens on the inner surface of the tubing with the nitric oxide from the polymer. At least a portion of the pathogens on the inner and/or outer surfaces of the tubing are inactivated (killed, disinfected, immobilized, neutralized, or otherwise reduced in virulence) via contact with the NO. For example, NO not only inactivates pathogens on the inner surface of the tubing, but NO can diffuse thought the wall of the tubing to inactivate bacteria on the tubing's outer surface. The pathogens contacted can include, for example, all kinds of bacterium, virus, fungus, and parasites. Exemplary aerobic and anerobic pathogens that can be inactivated using insert 1 include, for example, S. aureus, E. coli, S. epidermidis, P. mirabilis, S. mutans, P. aeruginosa, Klebsiella, C. albicans, MRSA, Veillonella, Actinomyces, Haemophilus, Neisseria, Vancomycin resistant enterococcus, Bacillus anthracis, Salmonella typhimurium, influenza virus, parasites like helminth S. mansoni, Leishmania donovani, and various mycobacterium. However, this list is not meant to be limiting-NO has broad spectrum activity and this is just a brief list of example pathogens that NO has been reported to kill.

The method examples given in this disclosure generally describe medical tubing and catheters. The methods are especially advantageous for indwelling catheters or tubing of extracorporeal medical devices. Example medical devices that may benefit from the use of insert 1 include, but are not limited to, intravenous catheters, urinary catheters, insulin cannulas, wound healing devices (e.g., wound dressing or wound patch), peritoneal dialysis catheters, hemodialysis catheter, feeding tubes, a photodynamic therapy device, intestinal tubes, cardiopulmonary bypass device, and the like. However, the methods are adaptable to tubing used in other industries. For example, a disinfection insert such as those described herein could be used to disinfect piping transporting non-biological fluids.

In some examples, the methods include fastening insert 1 to the end of the tubing. In some embodiments, insert 1 is replaceable. For example, the methods of use can include unfastening the first disinfection insert from the proximal end of the tubing and replacing it by fastening a second disinfection insert to the proximal end of the tubing.

The step of illuminating insert 1 can include activation of a light source that is in optical communication with insert 1. For example, insert 1 can include a fiber optic in optical communication with the light source. Illumination of the light source illuminates the fiber optic, which illuminates an NO donor within the polymer, which in turn releases the NO from the polymer. In some examples, the NO donor is a RNSO. Some methods of use include a step of attaching insert 1 to a coupling on a light source.

In some examples of the methods of use, NO is released from the polymer at a flux between 0.1×10⁻¹⁰ mol cm⁻² min⁻¹ and 100×10⁻¹⁰ mol cm⁻² min⁻¹. In some example methods of use, the intensity of light from the light source can be changed to vary a flux of NO from the polymer. In some examples, the wavelength of light from the light source can be changed to vary a flux of NO from the polymer.

In some embodiments, light source 3 can be controlled by wireless communication (for example, using a remote control, or a remote user interface on a computer or a mobile phone). For example, the light source can be controlled using software that acts as a light source controller (e.g., light source controller 18). As described above, light source controller 18 can, for example, be programmed to gradually increase light intensity so that the NO flux remains relatively constant over time until depletion. In some examples, the manufacturer or clinician has access to light source controller 18, to control the NO levels. The methods of use can include protections against patient tampering. The protections can be embedded within the light source controller software, for example. Furthermore, patients could be trained to replace the insert at certain time intervals in long-term situations.

### Examples

### Example 1: Materials and Methods

Materials: N-Acetyl-D-penicillamine (NAP), sodium nitrite, L-cysteine, sodium chloride, potassium chloride, sodium phosphate dibasic, potassium phosphate monobasic, copper (II) chloride, ethylenediaminetetraacetic acid (EDTA), tetrahydrofuran (THF), and sterile phosphate buffer saline powder with 0.01 M, pH 7.4, containing 0.138 M NaCl, 2.7 mM KCl, were purchased from Sigma Aldrich (St. Louis, MO). Methanol, hydrochloric acid, and sulfuric acid were obtained from Fisher Scientific (Hampton, NH). Helixmark® silicone tubing-silastic material (60-011-06) were purchased from VWR (Radnor, PA). A 12V 1.5 W LED light source (Rayauto) and LED BLE Bluetooth 4.0 software (Guixing Tan) were used for the light studies. All aqueous solutions were prepared using deionized water. Phosphate buffer saline (PBS) 0.01M with 100 Micromolar EDTA was used for all material characterization and NO analyzer studies. Dulbecco's modified Eagle's medium (DMEM) and trypsin-EDTA were purchased from Corning (Manassas, VA20109). The Cell Counting Kit-8 (CCK-8) was purchased from Sigma-Aldrich (St. Louis, MO). Penicillin-Streptomycin (Pen-Strep) and fetal bovine serum (FBS) were obtained from Gibco-Life Technologies (Grand Island, NY). The bacterial strains Staphylococcus aureus (ATCC 6538) and E. coli (ATCC 25922) were obtained from American Type Culture Collection (ATCC). All the buffers and media were sterilized in an autoclave at 121° C., 100 kPa (15 psi) above atmospheric pressure for 20 minutes prior to the biocompatibility experiments.

S-nitroso-N-acetvlpenicillamine Synthesis: The S-nitroso-N-acetylpenicillamine (SNAP) synthesis procedure was adapted from previously a published report with a slight modification. Briefly, NAP and sodium nitrite were taken in equimolar amounts and dissolved in a 2:3 ratio of water and methanol. To the above mixture, 0.7 and 1.6 M of H₂SO₄ and HCl were added, respectively followed by stirring for 10 minutes at room temperature. The beaker was shielded from ambient light, puffed with mild air, and incubated in an ice bucket for 8-10 hours. After incubation, filter paper was used to collect the SNAP crystals into a Buchner funnel using suction filtration. SNAP was then cleansed with ice-cold deionized water and placed in a vacuum desiccator overnight to remove the surplus solvent. Samples were shielded from light throughout the process and the purity of SNAP crystals (>90%) was verified using the chemiluminescence NOA and UV-vis calibration curve at 340 nm.

SNAP Impregnation: To impregnate the silicone rubber (SR) tube with SNAP, a stock solution of SNAP and THE (125 mg mL⁻¹) was prepared. A 3 cm long Helix silastic SR tube with an inner diameter of 0.058 inches was incubated in SNAP-THF solution for 24 hours in the dark at room temperature. After 24 hours, SNAP impregnated tube (SR-SNAP) was removed from the solution and dried overnight in a vacuum desiccator protected from light. All samples were cleaned with PBS to remove excess SNAP crystals from the outer surface and lumen of the impregnated tube before conducting any further experiments.

Determining weight percentage SNAP using UV-vis: The amount of SNAP impregnated in the SR-SNAP tube was quantified using a UV-vis spectrophotometer (Cary 60, Agilent Technologies). For this, first the mass of each SR-SNAP sample was recorded using an analytical balance (Mettler Toledo™ XS105DU, Columbus, OH). Each SR-SNAP sample was soaked in THF for 30 minutes to extract all the SNAP impregnated into the SR tube. The sample appeared clear after incubation in the THF, indicating all SNAP had been extracted into the THF. The SNAP extracted solution was evaluated by UV-vis spectroscopy at 340 nm wavelength. The molar absorptivity of SNAP in THE at 340 nm was determined to be 909 M⁻¹ cm⁻¹. The weight percentage (wt %) of SNAP loaded is reported as milligrams of SNAP loaded per milligram of tube.

Fabrication of the disinfection insert: A disposable catheter disinfection insert (DCDI) was produced using a 2.9 cm SR-SNAP tube mounted on a 7 cm segment of 1.5 mm diameter PMMA side glow optical fiber (Huaxi). As described herein, DCDI may be the same as, or functionally equivalent to, insert 1, described above. A layer of aluminum foil was wrapped around the rest of the optical fiber followed by a layer of parafilm to only allow light to emit from the SR-SNAP section of the DCDI. The DCDI samples were attached to a 12V 1.5 W LED light source (Rayauto) and controlled via Bluetooth using a mobile phone application.

Light emission spectroscopy measurements: To determine the wavelength of light emitted by LED light source, a wireless spectrophotometer (PS-2600, PASCO Scientific) with a detection range of 380-950 nm was utilized. The light detecting fiber optic cable was held in place with a clamp and DCDI samples were exposed to the detector and wavelength of light was recorded with four different colors (red, blue, green and white). Studies with light were done in the absence of ambient light to ensure only desired lights are being characterized.

Photoinitiated NO release: The NO release from the SNAP impregnated samples with light (SNAP-light) and without light (SNAP) was quantified using a gold standard chemiluminescence NO analyzer (NOA) 280i (Zysense, Frederick, CO) under nitrogen atmosphere. Experiments were performed at physiological temperature (37° C.) both in dry (to validate the effect of light on NO release) and in the presence of PBS (emulating the ‘real-world’ application). The NO release from the samples is normalized to the surface areas and presented as moles min⁻¹ cm⁻².

NO release vs. light color: In order to optimize the light from DCDI, samples were placed in an NOA sample cell at 37° C. connected to the LED light source. Using the mobile phone application, the LED light source was set to emit light at 100% intensity and the NO flux was recorded as the light color was adjusted. Four different light colors (white, blue, green, and red) were tested for their NO release.

24 Hour NO Release: The samples were immersed in PBS with EDTA (7.4 pH) at the physiological temperature of 37° C. in an amber NOA sample cell. For the SNAP-light samples, the LED light source was set to emit white light at 100% intensity. 24 hour NO release profiles for the DCDI samples were recorded using 0% (light off), and 100% white light intensity. The NO release was quantified by averaging the NO release during the first 2 hours of the experiment and at the 24-hour time point.

Tunable NO Release: The SNAP-light samples were placed in an NOA sample cell at 37° C. protected from ambient light. The LED light source was set to emit white light from the connected fiber optic and the NO flux from samples were recorded as the light intensity was adjusted using smartphone application. Starting without light (in dark), the intensity of light was increased in 25% increments until 100% intensity was reached. The light intensity was then decreased by 25% until 0% to show the strong control over NO levels. The NO release for each step was allowed to plateau before changing to the next step.

Determination of SNAP leaching: The amount of SNAP leached from samples SR-SNAP (light off) and SNAP-light (100% light intensity) was determined by a UV-vis spectrophotometric method. Each sample was incubated in 10 mM PBS, pH 7.4, with 100 microMolar EDTA at 37° C. for 24 hours. The soaking buffer was evaluated for SNAP concentration at 2, 4-, 6-, 8- and 24-hour timepoints. The molar absorptivity of SNAP in 10 mM PBS, pH 7.4, with 100 Micromolar EDTA at 37° C. was determined as 1072 M⁻¹ cm⁻¹ at 340 nm. Samples were stored at 37° C. throughout the duration of the experiment. Results were analyzed by calculating SNAP concentration from each sample, normalized by the surface area of DCDI.

In Vitro Antibacterial Properties: 4 hour Bacterial Adhesion Assay: The antibacterial activity of DCDI was examined against S. aureus and E. coli in terms of viable bacterial adhesion on the catheter surface. All samples (SR, SR-light, SNAP, and SNAP-light; n=3 each) were exposed to the bacterial solution with the final OD600 of bacteria ranging between 10⁶-10⁸ CFU mL⁻¹ for 4 h at 150 rpm at 37° C. to maintain the chronic infection conditions. After 4 hours, bacteria adhered on the DCDI surface were extracted, diluted, and plated using a spiral plater (Eddy Jet 2, IUL Instruments) (50 microLiters). Viable colony forming units (CFU) were determined after 24 hours of incubation at 37° C. using an automated bacteria colony counter (Sphere Flash, IUL Instruments). The CFU's on the DCDI (SNAP-light) and controls were normalized by surface area of the samples and percentage of reduction in bacterial viability was determined by the following equation.

$$\% bacterial reduction = \frac{\begin{matrix}( C F U {cm}^{- 2} on SR ) - \\ ( C F U {cm}^{- 2} on SNAP - light ) \times 100\end{matrix}}{( C F U {cm}^{- 2} on SR )} \qquad Equation 1$$

In Vitro Ability of DCDI to treat established microbial infections on catheter surfaces: To evaluate the efficacy of DCDI in disinfecting the infected catheter, an in vitro assay was developed using S. aureus bacteria. S. aureus was grown overnight in an LB media following the same procedure as the 4-hour bacterial adhesion assay, above. Then, 3 mL of bacterial suspension (0.1 OD) was used to expose the model CVC catheter (Dow Corning 60-011-09 Helix silicone tubing) 3 cm long; such that both inner lumen and outer surface of the catheter were exposed to bacteria. Samples were exposed in the media for 24 hours in LB medium at 37° C. (static conditions). Nutrient media in the tubes was replaced periodically to keep the supply of nutrients stable. After 24 hours, samples were taken out from media containing bacteria and briefly rinsed to remove any unadhered cells on the catheter tubing. Later, DCDI samples with 100% white light intensity and control samples were then inserted into the infected catheter tubing and incubated in PBS pH 7.4 for 4 hours at 37° C. under static conditions. The control samples contained no NO-releasing DCDI, just a plain SR tubing mounted on fiber optic inserted into the bacteria infected catheter. Samples were taken out after incubation and bacteria remaining on the catheter surface were enumerated using the same protocol as the 4-hour bacterial adhesion assay, above.

In Vitro Cytocompatibility Study: Leachate Preparation: All test and control groups SR, SNAP, light, and SNAP-light samples (n=3) each were first cleaned with ethanol and UV-sterilized for 30 minutes. Next, DMEM media (1 mL) was added to the samples to collect the leachates in the solution by following the ISO standards (ISO10993-5:2009 Test for in vitro cytotoxicity). Vials were covered in aluminum foil to prevent from ambient light and incubated for 24 hours at 37° C. After 24 hours, the samples were removed, and the leachates were used for the further analysis.

In Vitro Cytocompatibility Study: Cell Viability: A cell culture treated 96-well plate was used to seed 3T3 mouse fibroblast cells (5000 cells/mL) in each well and incubated in a humidified incubator at 37° C., 5% CO² for 24 hours. Later, leachate samples were exposed to the cells (100 microLiters) and incubated for another 24 hours to let the leachates act on the cells. The cytocompatibility study was conducted in compliance with the ISO 10993 standard using a CCK-8 cell viability kit following the manufacturer's instructions (Sigma, OH). To each of the wells CCK-8 solution was added (10 microLiters) and incubated for 1 hour. The absorbance (abs) of the samples was recorded at 450 nm wavelength using microplate reader (Cytation 5 imaging multi-mode reader, BioTek). Results from the experiment are reported as relative cell viability of test group normalized to control (cells in media) using the following equation:

$$Relative cell viability = \frac{absorbance test group}{absorbance control group} \qquad Equation 2$$

Statistical Analysis: All results in the study are presented with sample size n≥3. Data are all reported as mean±standard error of mean (SEM). Statistical significance between the sample types was determined using student's t-test. To ascertain the significance of results, value of p<0.05 was used to draw the comparison between the test (light, SNAP, SNAP-light) and control groups (SR).

Sterilization of SNAP impregnated tubing: Sterilization of medical devices is an important process for decontaminating the surfaces before in vivo application. Ethylene oxide and ultraviolet light sterilization methods were tested on the DCDI. For ethylene oxide sterilization, the NO releasing insert were packaged into the sterilization pouch and exposed to EO under AN 74i Anprolene EO gas sterilizer (Anderson Sterilizers). The sterilizer was operated at room temperature (between 68-91° F.) with a Humidichip to ensure at least 35% humidity was achieved. Samples were sterilized for 12 hours with 2 hours of purging. For UV light sterilization, SR-SNAP samples were sterilized with UV-light under biosafety cabinet administered by REDISHIP Purifier® Logic®+Class II A2 Biosafety Cabinets, Labconco® for 30 minutes. All the samples (n=4) were pre-weighed and suspended in THE (30 minutes) to extract all the SNAP from the polymer. The amount of SNAP remaining in the SR-SNAP tubing after sterilization process was compared to fresh samples using UV-vis spectroscopy by measuring the absorbance of SNAP extracted in THE at 340 nm wavelength. The results of the study are reported as normalized values to initial weight percent of SNAP (weight of SNAP/weight of polymer).

### Example 2: Fabrication Results

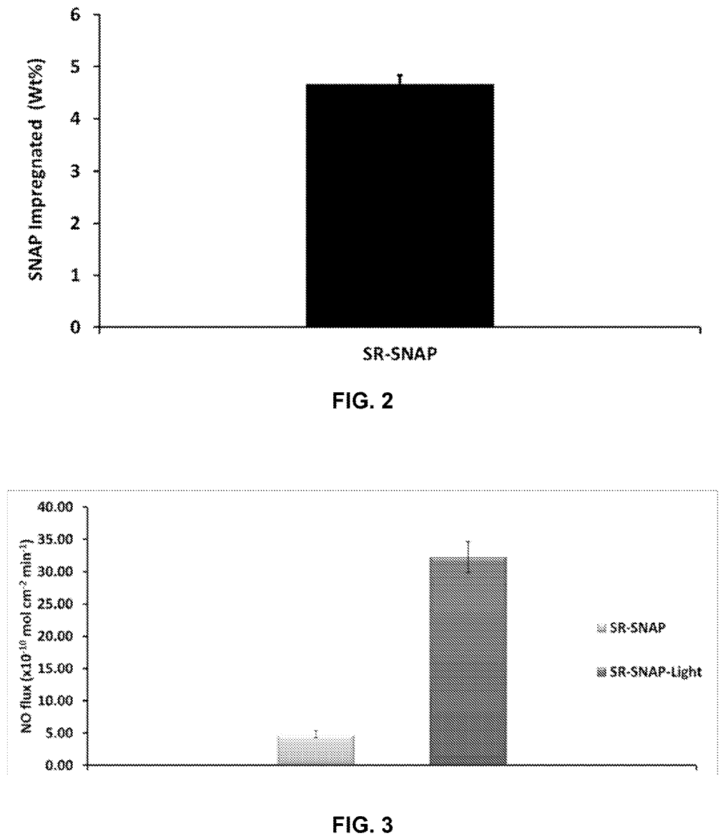

It has been previously reported that SNAP incorporation/impregnation into various polymers, like polyurethanes and silicone elastomers, gives long-lasting and controlled NO release, with enhanced shelf-life stability, ability to withstand sterilization, low rates of SNAP leaching, and photosensitivity. The wt % SNAP impregnated in the SR-SNAP samples was quantified using a UV-vis spectrophotometer. The solubility of SNAP in THE allowed the full extraction of the impregnated SNAP in each sample. As shown in FIG. 2, the results indicate that 4.66±0.16 wt % of SNAP was impregnated into the SR-SNAP samples. The values obtained here agree with the previously reported values for SNAP impregnation of SR that accomplished ≈ 5 wt % with the same concentration of SNAP-impregnation-solvent (125 mg mL⁻¹). 24 hours of soaking SR in SNAP-tetrahydrofuran (THF) solution (125 mg mL⁻¹) resulted in 4.66±0.16 wt % SNAP loading. Data represent mean±standard error of mean (SEM) for n≥3.

### Example 3: Determining the Wavelength of Light Emitted by LED Light Source

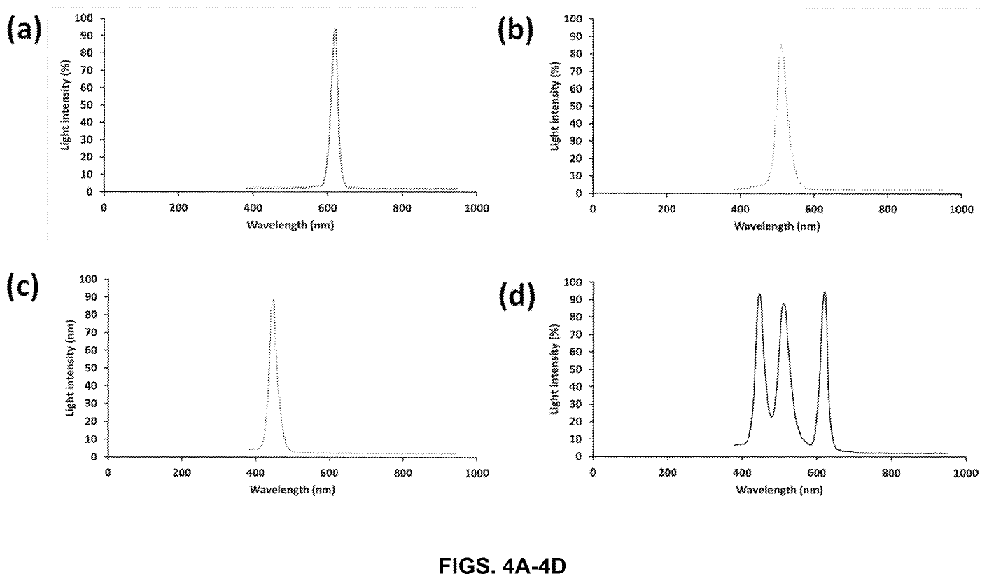

To confirm the wavelength of light emitted by the LED source, a PASCO wireless spectrometer optical probe was utilized. The wavelength of light was determined at four different colors of light. As shown in FIGS. 4A-4D, the test results confirm that the red, green, and blue light had emissions ranging from 570-650 nm, 475-575 nm, 450-500 nm, respectively (FIGS. 3A-3D). In particular, FIGS. 4A-4D show the light intensity of red (621 nm), green (512 nm), blue (447 nm), and white (e.g., a mixture of red, green, and blue) set at 100% light intensity, according to some embodiments. Furthermore, the study confirmed the white light provided by the light source is comprised of red, blue, and green lights.

### Example 4: NO Release Vs. Light Color

Side glow fiber optic is thin, flexible, and illuminating. Side glow fiber optic was chosen over end glow fiber optic to illuminate the full length of SNAP tube, ensuring the effectiveness of eradicated bacteria along the entire length of DCDI inserted within a catheter. The thin, flexible nature of the fiber optic and a tubular scattering factor enables the light to uniformly disseminate through the catheter lumen.

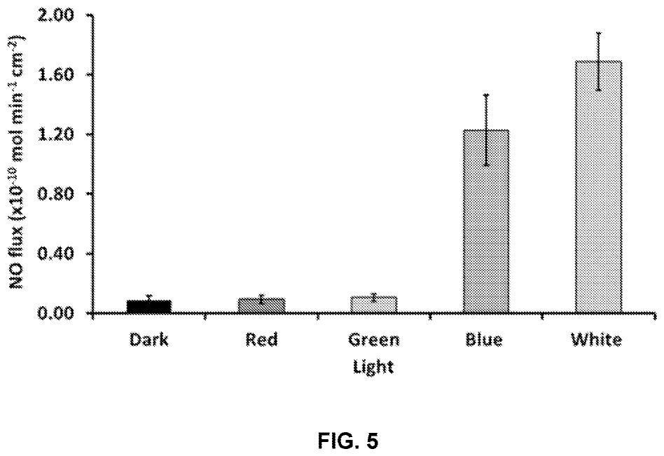

Referring now to FIG. 5, a comparison of NO release from DCDI at physiological temperature (e.g., 37° C.) in the dark and photoinitiated at 100% light intensity of red (620 nm), green (530 nm), blue (450 nm), and white (mixture of red, green, and blue) light is shown, according to some embodiments. NO release from DCDI was triggered highest at 100% white light. Data represent mean±SEM (n≥3). Specifically, to optimize the light color for the study, NO release from the DCDI was tested against various colors of light (red, green, blue, and white) at 100% light intensity (n≥3). DCDI samples were inserted in the amber NOA cell to protect the samples from ambient light. First, the NO release from the samples was recorded in the dark. Then, using the mobile application, the color of the light was changed, and the intensity was adjusted. Interestingly, while the NO release in dark was 0.09×10⁻¹⁰ mol cm⁻² min⁻¹ flux, the red, green, blue, and white light at 100% light intensity triggered 0.14, 0.17, 1.23, and 1.69×10⁻¹⁰ mol cm⁻² min⁻¹ of NO from DCDI, respectively, as shown in FIG. 5. Since the levels of NO were seen to be higher with the trigger of white light, all the further studies were conducted with white light.

Real time control of NO Release: It would be useful to be able to dynamically control NO level depending on the biomedical application. For instance, at the time of implantation, the catheter may need a higher level of NO to prevent bacterial colonization on the device surface. However, over time the same device may need lower levels of NO to maintain the biofilm-free state of the device. Similarly, a significantly contaminated device surface may require very high levels of NO to disinfect and eradicate the pre-established biofilm on the catheter surface. Tight control of NO release can be achieved by exploiting the photosensitivity of NO donating compounds that can tune the NO release in response to intensity and wavelength of light.

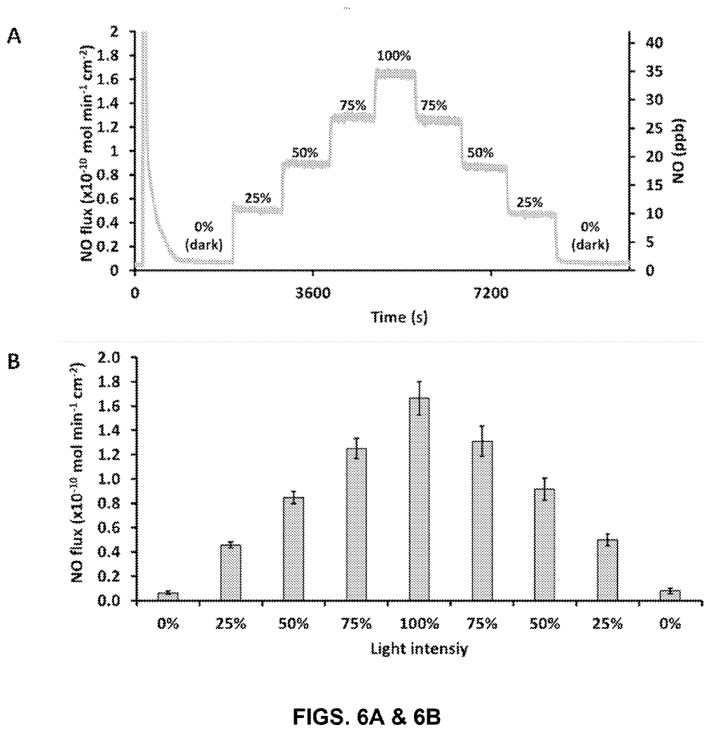

The ability to control the release of NO by changing the light intensity was assessed by increasing and decreasing the white light intensity in 25% intervals. To avoid the interference of leached SNAP in buffer conditions, a dry state at physiological conditions was used to study the influence of white light on DCDI at different light intensities. Referring generally to FIGS. 6A and 6B, the results from the study showed each degree of light intensity can steadily trigger the DCDI to release NO at a particular level which can be modulated real time. In particular, FIG. 6A shows NO release measured at increasing and decreasing intensities of light (0%, 25%, 50%, 75% and 100%) changing at 15-minute intervals. FIG. 6B shows the quantification of NO release using chemiluminescence measured with the trigger of different light intensities at 37° C. Data represent mean±SEM (n≥3). The amount of NO released at each light intensity are tabulated in Table 1. The advantage of this approach is that the NO release levels from the DCDI can be modulated via adjusting the intensity of the light source connected to the fiber optic, the specific NO donor employed in the DCDI design, and the amount of the NO donor incorporated in polymeric tube. Making use of light as a trigger to potentiate the NO release enables accurate monitoring of turning NO on and off in addition to increasing or decreasing NO flux as required. This provides a precise analysis of regulating NO levels required to achieve antibacterial properties. In this study, the NO donor molecule SNAP is utilized in the DCDI, but other light-sensitive NO donor molecules could similarly be employed (for example various S-nitrosothiols, modified S-nitrosothiols, or combinations of various NO donor molecules).

TABLE 1
NO release levels measured from DCDI at different light
intensities at 37° C. using chemiluminescence nitric
oxide analyzer. Data represents mean ± SEM (n ≥ 3).

| | NO flux (×10⁻¹⁰ mol |
|---|---|
| Light intensity | min⁻¹ cm⁻²) |
| 0% | 0.06 ± 0.01 |
| 25% | 0.46 ± 0.03 |
| 50% | 0.85 ± 0.05 |
| 75% | 1.25 ± 0.08 |
| 100% | 1.66 ± 0.14 |
| 75% | 1.31 ± 0.13 |
| 50% | 0.92 ± 0.09 |
| 25% | 0.50 ± 0.05 |
| 0% | 0.08 ± 0.02 |

Real time NO Release: To confirm if the DCDI could be applied to disinfect in the buffer environment, the NO release from the DCDI was measured under physiological conditions (37° C. in PBS buffer) using chemiluminescence for 24 hours at 0% (dark), and 100% white light intensity. Results confirmed that DCDI submerged in PBS could still be set alight via the mobile phone application for at least 24 hours, at levels equivalent to the data presented in FIGS. 5A and 5B. The SNAP impregnated SR has a fixed amount of NO and will exhaust over time as NO is constantly discharged. The 100% light intensity significantly boosts the rate at which NO is depleted from the samples early in the experiment, which might explain the marginally lower flux of NO released at the 24 hour time point.

Although the testing was done only up to 24 hours, reports have shown that SNAP loaded SR can have prolonged NO release. Overall, the DCDI was able to closely simulate the levels of NO released by endothelium (0.5−4×10⁻¹⁰ mol cm⁻² min⁻¹) in the body which is responsible for important biological functions such as reducing inflammation and fibrosis, killing various microbial species (bacteria, fungus, viruses), inhibiting disrupting and dispersing microbial biofilm formation, preventing platelet activation, reducing clotting and thrombosis. The NO release levels achieved here corroborate with previously reported studies that include impregnation of silicone-based polymers for biomedical applications. Nitric oxide in the gaseous form is known to have a very short half-life. NO donors blended, conjugated, or impregnated in the polymer matrix for therapeutic and targeted NO release exhibit exceptional stability and biocompatibility. Wo et al. have shown the stability of SNAP during storage in different polymeric matrix that revealed the stability of SNAP for up to 8 months. The excellent storage stability can be attributed to the intramolecular hydrogen bonding between the SNAP crystals within the polymer-crystal composite.

### Example 5: Quantification of SNAP in Soaking Buffer

RSNO's have been known to decompose via heat, light, metal and chemical-based mechanisms, all of which exhibit catalytic activity that trigger NO release from the donors. Even today, RSNO-based polymeric devices have a major limitation due to the leaching of NO donor which not only compromises the duration of NO delivery but also at times can result in unfavorable body reactions. Impregnating the NO donor SNAP into hydrophobic polymers like SR has been demonstrated to regulate leaching, which consequently prolongs the NO release from the polymer. Due to the intramolecular hydrogen bonding between SNAP molecules and the low water uptake of hydrophobic polymers, SNAP dissolution and dissemination out of the polymer is highly contained.

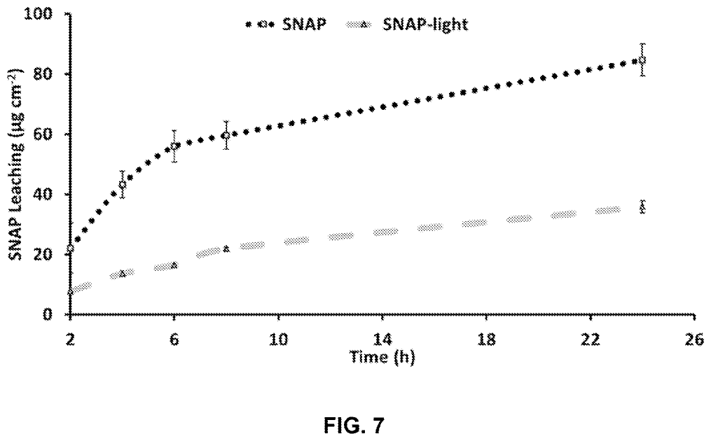

To quantify the amount of SNAP leached, both SNAP (dark 0% light) and SNAP-Light (100% white light) samples were soaked in PBS-EDTA at 37° C. for 24 hours. The soaking buffer was collected at 2, 4, 6, 8 and 24-hour timepoints and absorbance was recorded using UV-vis spectroscopy. After 24 hours, 84.61±5.32 and 35.83±2.04 micrograms cm² of SNAP was detected from SNAP and SNAP-light samples, respectively (FIG. 7). In FIG. 7, data is shown normalized to the polymer surface area and represented as mean±SEM (n=3). It is possible that introduction of light to SNAP samples (SNAP-light) significantly enhanced the catalysis rate of NO from SNAP. As a result, lower amount of SNAP is detected in the soaking buffer for these samples as compared to SNAP only samples.

Lower quantities of SNAP leach out of the polymer and eventually degrade into N¬-acetyl-D-penicillamine (NAP), and NAP-dimer. Any minimal leaching of SNAP, or more likely NAP (N-acetyl-penicillamine) and dimers of NAP, ultimately hydrolyze to penicillamine. Low levels of penicillamine is not a major concern since it is an FDA approved agent used to treat heavy metal poisoning in humans. Besides, studies in the past have demonstrated SNAP to be safe at low concentrations during in vivo testing of SR-SNAP. The advantage of introducing light to the SNAP impregnated polymer is the NO release from the SNAP can be amplified by enhancing the catalytic rate of NO from the NO donating compound. As the leaching reduced, the corresponding NO release rate increased highlighting the advantages of light.

### Example 6: Evaluating the Antibacterial Efficacy of DCDI

Over 1 million cases of hospital acquired infections are reported every year. About 60-70% of these infections arise from bacterial contamination and biofilm formation on the surface of medical devices, which severely compromises the durability of the medical devices and associated cost. With time, biofilm-associated pathogens are acquiring more and more resistance mechanisms against standard antibiotic treatment, leaving the conventional treatments futile. For this reason, many novel antimicrobial approaches have been proposed. Light based antimicrobial therapy is one such strategy being employed for combating biofilms on medical implants. Clinical pathogens such as S. aureus and E. coli have been proven to be vulnerable to photodynamic inactivation by the wavelengths of visible spectrum of light (400-800 nm). Similarly, photoactivation of silver and gold nanoparticles have also been explored for bactericidal efficacy. However, metal-dependent disinfection can vary by the type of metal, and for Gram positive versus Gram negative bacteria. Furthermore, metal-dependent disinfection has some cytotoxicity towards mammalian cells. For this reason, a broad-spectrum, biocompatible disinfecting device was developed in this study for eradicating both Gram-positive and Gram-negative bacteria.

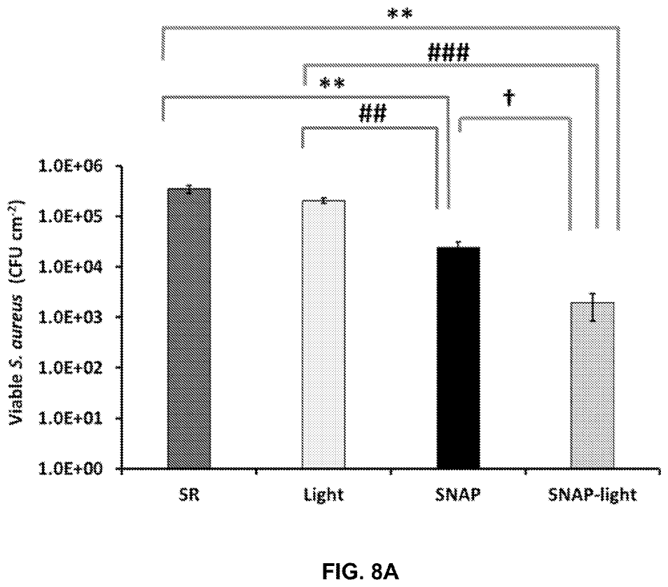

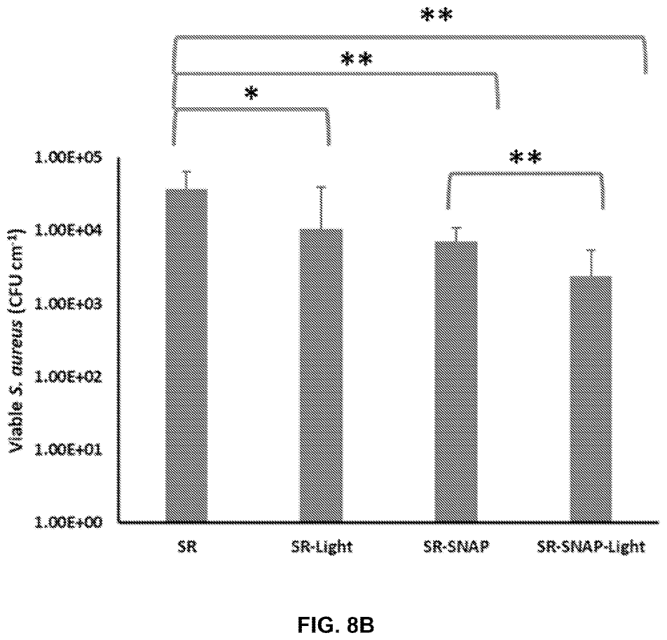

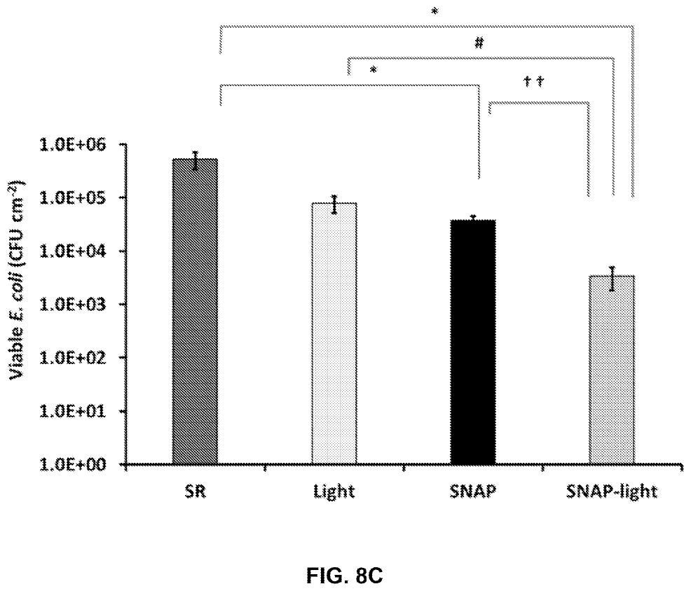

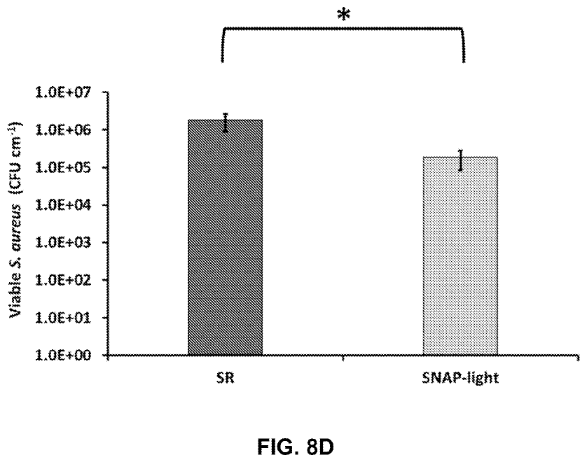

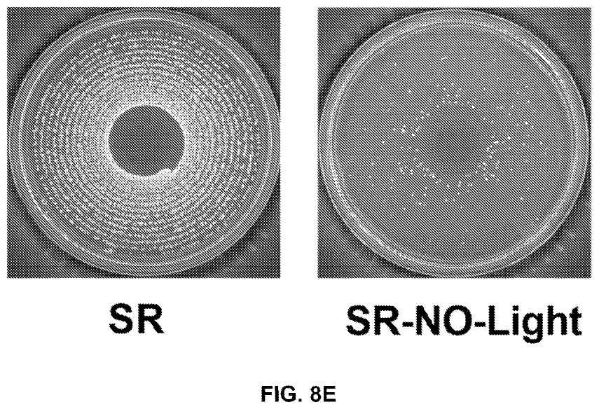

The bactericidal efficiency of DCDI was evaluated against S. aureus and E. coli bacteria using a 4 hour bacterial adhesion assay. The bacterial cells adhered on the DCDI were enumerated and normalized to the surface area of the catheter to obtain viable CFU cm⁻². Results from S. aureus adhesion on the synergy of SNAP-light unveiled an approximate 99.45% reduction compared to the SR control (p<0.05). As shown in FIG. 8A, the SR-light and SNAP were observed to have about 41.30% and 93.05% reduction, respectively, in terms of viable adhered cells (p<0.05) due to the action of the NO release and light mediated interface individually. In FIG. 8A, data represent mean±SEM (n≥3), ** represents p≤0.01, calculated for SNAP, SNAP-light vs. SR, ##represents p≤0.01, calculated for SNAP vs. light, ###represents p≤0.001, calculated for SNAP-light vs. light, t represents p≤0.05 calculated for SNAP-light vs. SNAP. The reduction in viable S. aureus is also shown in FIG. 8E, which includes an example agar plate CFU after 4 h of exposure to a control insert (e.g., a non-impregnated silicon rubber (SR) sample) and insert 1 (e.g., a SR—NO-Light DCDI) in the in situ catheter disinfection model.

Referring also to FIG. 8B, results of a second test are shown. In this example, the bacterial cells adhered on the DCDI after a 2 h exposure were enumerated and normalized to the length of the sample to obtain viable CFU cm¹. Results from S. aureus adhesion on the synergy of SNAP-Light unveiled a ca. 93.62% reduction compared to the SR control (p<0.05). In FIG. 8B, ** represents p≤0.01, calculated for SR-SNAP, SR-SNAP-light vs. SR, * represents p≤0.05, calculated for SR-Light vs. SR. The SR-Light and SR-SNAP were observed to have ca. 71.91% and 81.15% reduction, respectively, in terms of viable adhered cells due to the action of the NO release and light mediated interface individually. These results demonstrate the potent and rapid antimicrobial activity of the covalently immobilized SNAP-PDMS DCDI, which has potential applications to eradicate infection on a wide range of indwelling medical devices (e.g., catheters, endotracheal tubes).

While the light and SNAP individually led to reductions of about 85.01 and 92.89%, results from the E. coli study showed highest reduction in viable E. coli adhesion on the surface of SNAP-light DCDI at 99.36% compared to SR control (p<0.05), as shown in FIG. 8C. In FIG. 8C, Data represent mean±SEM (n≥3), * represents p≤0.05, calculated for SNAP, SNAP-light vs. SR, #represents p≤0.05, calculated for SNAP-light vs. light, †† represents p≤0.01 calculated for SNAP-light vs. SNAP. Overall, the synergistic effect of SNAP and light helped in significant inhibition of both S. aureus and E. coli bacteria on the surface of the DCDI. Chemical alteration of the DNA by reactive nitrogen species is one of the leading means of NO enabled antimicrobial action. NO's reaction with oxygen and peroxides leads to formation of range of antimicrobial species such as peroxynitrite and nitrogen dioxide that can alter and destroy the DNA base pairs. The damage of DNA strands facilitates lipid peroxidation, constrains enzyme functions and results in eventual membrane loss in microorganisms.

### Example 7: Efficacy of the DCDI to Treat Established Infections on Catheter Surfaces

The ability of DCDI to eradicate pre-colonized bacteria on a catheter surface was characterized against S. aureus, one of the most common bacteria associated with biofilm-related infections. The S. aureus bacteria was grown till mid-log phase and exposed to model CVC catheters for 24 hours at 37° C. After 24 hours, the DCDI and the corresponding SR control was then inserted into the infected catheter to investigate the disinfecting ability. Viable bacteria remaining on the surface of the catheter were enumerated and reported as CFU cm⁻¹ of the polymer. Referring to FIG. 8D, the study showed SNAP-light DCDI could eradicate S. aureus by ca. 96.99% compared to the plain SR control (p<0.05). In this example, the intravascular catheters were first exposed to S. aureus for 24 hours to infect the catheter surface with biofilm, followed by a 4-hour treatment with the NO-releasing disinfection insert (SNAP-light). Antimicrobial activity is calculated as the log of the colony forming units (CFU) cm⁻² of polymer surface area; data represents mean±SEM (n≥3).

Nitric oxide is known to induce biofilm dispersal across many bacterial strains, which led to its importance in emerging as therapeutic for biofilm-related infections. NO is a reactive gas with very short half-life with an ability to diffuse through the cell membranes spontaneously. Previous reports have shown that NO at lower concentrations can trigger the switch of sessile cells to free floating planktonic phenotype in bacterial cells enclosed within the biofilm. It is understood that the control of intracellular secondary messenger such as cyclic di-GMP by NO imitates the effectors which can hamper the biofilm buildup and disperse the mature biofilm. The reactive nitrogen species from NO and the superoxide ions lower the extracellular polysaccharide production which is an important intermediary component for bacterial attachment on a substratum. The role of NO in facilitating biofilm dispersion is maintained across a wide range of bacterial species.

The results from prevention of bacterial adhesion (FIGS. 8A-8C) and disinfection of catheter with pre-colonized bacteria (FIG. 8D) both support the significance of DCDI in treating and preventing catheter infections in clinical settings. Light induced DCDI is a potent and locally acting antimicrobial device that is biocompatible, low cost, shelf stable and easy to apply. These characteristics makes it a favorable choice for integrating with medical devices such as with long-term usage for decontaminating them in between uses. Therefore, development of DCDI is expected to be a significant step towards preventing microbial contamination in both short- and long-term indwelling catheters that are regularly at a higher risk of contracting an infection. The DCDI can be applied to wide range of catheter devices including intravenous catheters, urinary catheters, insulin cannulas, wound healing devices, peritoneal dialysis catheters, hemodialysis catheter, etc.

### Example 8: Cytocompatibility of DCDI

Over the years, eukaryotic cells have developed mechanisms for scavenging the reactive oxygen and nitrogen species which enables them to negate their influence; however, various microorganisms (bacteria and virus) remain vulnerable. The combination of SNAP and light was effective in lowering the number of viable microbial cells. Nonetheless, it is important to determine the compatibility of engineered disinfection insert towards mammalian cells for effective in vivo application. The biocompatibility of the DCDI and respective controls was verified using NIH 3T3 mouse fibroblast cells. To investigate this, SR, SNAP, light and SNAP-light DCDI samples were first incubated in DMEM media to collect the leachates in the solution. Later, the leachates were added to the cells and incubated for 24 hours at 37° C. to determine toxicity of the solutions against mouse fibroblast cells. After 24 hours, a cell cytotoxicity assay was conducted on the cells using a CCK-8 cell viability kit. Samples were read for their absorbance and data was analyzed to compare the absorbance of controls and test groups. Mouse fibroblast cells demonstrated no significant difference in the presence of the leachates from all the samples with respect to controls.

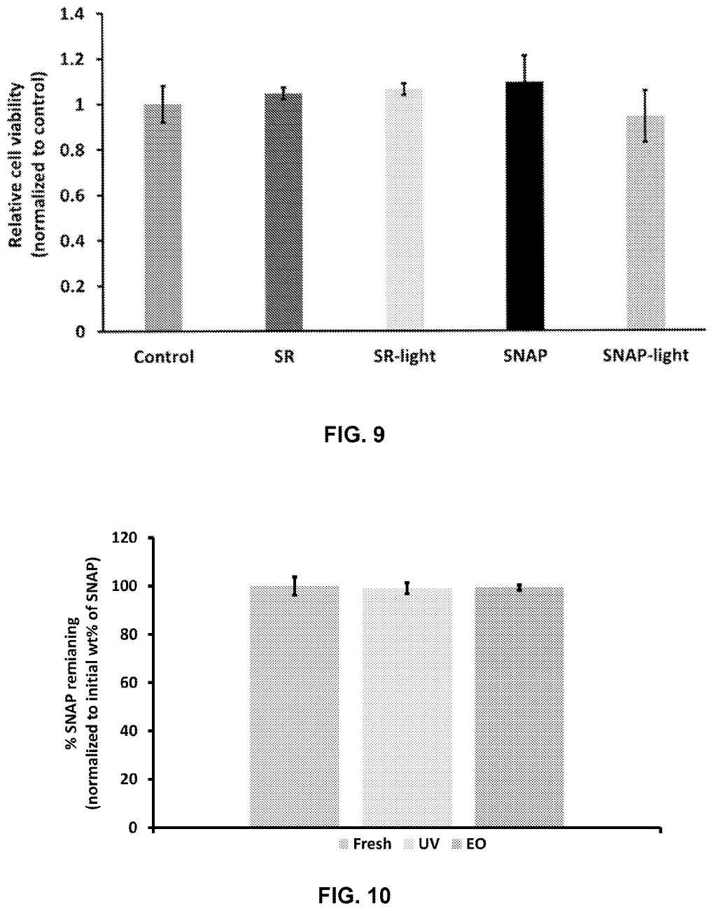

Referring now to FIG. 9, all the samples exhibited >90% viability in the cells over 24 hours. In this example, data represent mean±standard deviation (n≥3) reported as relative cell viability compared to control. Similar experimental designs have been previously used to demonstrate the biocompatibility of NO releasing polymers. The SNAP loaded NO-releasing polymers have been previously reported to be biocompatible both in vitro and in vivo. Such NO releasing medical grade polymers have also been shown to lower the platelet activation and thrombus formation in an in vivo rabbit model. Together these results from the cytocompatibility study offer encouraging evidence toward the potential biocompatibility of the light induced DCDI.

### Example 9: Sterilization of SNAP Impregnated Tubing

Medical devices must be sterilized before in vivo application, and so they must be able to withstand sterilization processes without compromising desired properties. NO donating compounds such as RSNO's are known to degrade due to their sensitivity to temperature and thermal degradation. It is therefore important to evaluate the compatibility of SNAP polymer with regularly used sterilization processes. To investigate their stability under sterilization process, samples were exposed UV-light and EO gas. Both sterilization methods were proven to be compatible with the NO releasing polymer. As shown in FIG. 10, results from the study indicate that 99.06±2.26% and 99.33±1.08% SNAP was conserved in the polymer after UV and EO sterilization, respectively. In this example, the retention of SNAP in the polymer after sterilization process was analyzed by extracting the SNAP remaining in the polymer in THE solvent and measuring the absorbance of SNAP at 340 nm using UV-vis. Data represents mean±SEM normalized to initial wt % of SNAP in freshly prepared samples (n=4). The ability for SNAP impregnated polymer to be sterilized with traditional methods without the loss of compound makes DCDI a suitable material for clinical translation.

### Example 10: Alternative Materials and Methods

Since SNAP is a small molecule and it has the tendency to leach out from polymer matrix over time, an alternative method of conjugating PDMS polymer to SNAP used to coat the fiber optic device is also described herein. Using the conjugated SNAP-PDMS, in this study, a battery-operated fiber optic light source (e.g., light source 3) was used to trigger the NO release from device. In some embodiments, the fiber optic light source emits a blue light in 450 nm wavelength spectra.

SNAP-PDMS synthesis: Hydroxy-terminated polydimethylsiloxane (PDMS-OH, 2550-3750 cst, 800 mg) is dissolved in anhydrous toluene (5 mL), which is then supplemented with (3-aminopropyl) trimethoxysilane (APTMS, 150 mg) and dibutyltin dilaurate (DBTDL, 3.4 μL). The solution is left stirred overnight. N-acetyl-D,L-penicillamine thiolactone (NAPTH, 150 mg) was then added to the reaction vessel which was stirred for an additional 24 h. Afterwards, the reaction mixture was nitrosated using t-butyl nitrite (1.2 mL) that was first purified with Cyclam to remove copper stabilizing agents. This final nitrosated solution was used for subsequent dip-coating processes, as described below.

Fabrication of second-generation disinfection insert: The NO-releasing fiber optic was developed by coating approximately 3 cm of the 10 cm long LC-connectorized Corning® Fibrance® Light-Diffusing Fiber Optic. Each sample was dip-coated with SNAP-PDMS solution five times with 1 min interval between each topcoat. The coated samples were allowed to air dry overnight at room temperature and then additional 24 h in vacuum desiccator to ensure all the solvent is evaporated from the samples. The NO-releasing samples were connected to single color light source 3 with continuous light mode for all the study. For control samples either PDMS or SNAP-PDMS were coated on fiber optic samples and operated with or without light resulting in the control samples of SR, SR-Light and SR-SNAP, respectively.

Controlling NO release from SNAP-PDMS coated insert: The nitric oxide (NO) release from SR-SNAP-Light was determined using a Zysense Nitric Oxide Analyzer 280i (NOA) which is the gold standard chemiluminescence detection system for measuring NO release in real-time. The NO released from samples was detected by the NOA in parts per billion and the flux values (moles min-1 cm-2) were normalized with the surface area of the sample. The samples were immersed in PBS with EDTA (7.4 pH) at the physiological temperature of 37° C. in an amber NOA sample cell. The ability to control the NO release from SR-SNAP-Light samples was demonstrated with light off and light on (continuous light mode) at 5 min interval. A similar study was done to evaluate the NO release from SR-SNAP and SR-SNAP-Light samples.

Antibacterial activity: The antibacterial activity of the samples was evaluated against S. aureus bacteria using a 2 h antibacterial adhesion assay. For this single isolated colony of S. aureus bacteria was inoculated in LB media and grown to mid-log phase. All samples (SR, SR-light, SNAP, and SNAP-light; n=3 each) were exposed to the bacterial solution with the final OD600 of bacteria ranging between 10⁶-10⁸ CFU mL⁻¹ for 2 h at 37° C. to maintain the chronic infection conditions. After 2 h, bacteria adhered on the surface were extracted, diluted, and plated using a spiral plater (Eddy Jet 2, IUL Instruments) (50 μL). Viable colony forming units (CFU) were determined after 24 h of incubation at 37° C. using an automated bacteria colony counter (Sphere Flash, IUL Instruments). The CFUs on the samples were normalized by the length of the samples and percentage of reduction in bacterial viability was determined by Equation 1, above.

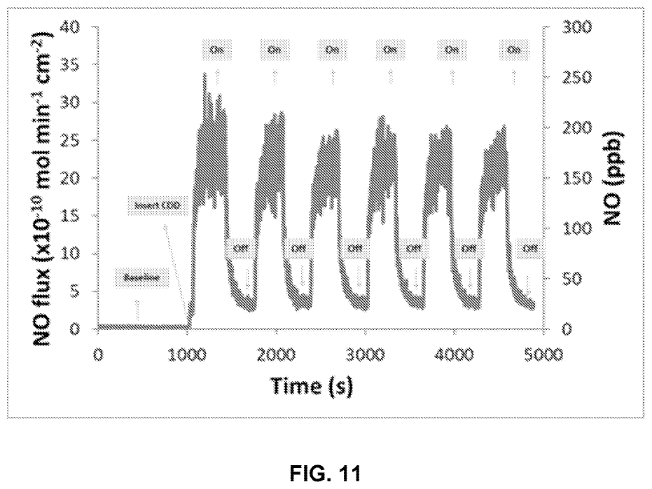

NO release properties: The ability to tightly control the NO release from the insert, fabricated with the covalently immobilized SNAP-PDMS polymer, was tested to evaluate the ability to tune and control the NO release using photoactivation by illuminating the side glow fiber optic. The light from the samples was turned on and off at 5 min interval. As shown in FIG. 11, results indicate that samples in dark released 3.45×10⁻¹⁰ mol cm⁻² min⁻¹ whereas turning the light on in continuous light mode resulted in 22x×10⁻¹⁰ mol cm⁻² min⁻¹. In this example graph, the NO release from a sample (e.g., insert 1) was measured using a nitric oxide analyzer (NOA) in PBS with 100 μM EDTA at 37 C. Thus, FIG. 11 clearly shows that NO release is greatly increased when light source 3 is turned on. For example, approximately 175 ppb of NO are released when light source 3 is turned on versus about 20 ppb when light source 3 is off. In this example, the light from the samples was turned on and off (i.e., modulated) at 5 min interval. In some embodiments, light source 3 is modulated (e.g., turned on/off) by light source controller 18, as described above.

The NO release from SR-SNAP and SR-SNAP-Light samples was tested by submerging the samples in PBS with EDTA, as shown in FIG. 3. The SR-SNAP samples (without light) demonstrated 4.78×10⁻¹⁰ mol cm⁻² min⁻¹ of initial NO flux. Notably, introduction of light to SR-SNAP-Light samples accelerated the NO released from these samples with 32.24×10⁻¹⁰ mol cm⁻² min⁻¹ of NO flux over 8 h. This data demonstrates the advantages of covalently immobilizing the NO donor functionality to the polymer, eliminating NO donor leaching and resulting in the high levels of localized NO release observed herein.

Antibacterial activity: The bacterial cells adhered on the insert after a 2 h exposure were enumerated and normalized to the length of the sample to obtain viable CFU cm⁻¹. Results from S. aureus adhesion on the synergy of SNAP-Light unveiled a ca. 93.62% reduction compared to the SR control (p<0.05), as shown in FIG. 8B, described below. The SR-Light and SR-SNAP were observed to have ca. 71.91% and 81.15% reduction, respectively, in terms of viable adhered cells due to the action of the NO release and light mediated interface individually. These results demonstrate the potent and rapid antimicrobial activity of the covalently immobilized SNAP-PDMS DCDI, which has potential applications to eradicate infection on a wide range of indwelling medical devices (e.g., catheters, endotracheal tubes).

### Configuration of Exemplary Embodiments

The preceding description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The implementation was chosen and described in order to best explain the principles of the invention and the practical application, and to enable others of ordinary skill in the art to understand the invention for various implementations with various modifications as are suited to the particular use contemplated.

For purposes of this description, certain aspects, advantages, and novel features of the aspects of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the operations of exemplary aspects of the disclosed method may be described in a particular, sequential order for convenient presentation, it should be understood that disclosed aspects can encompass an order of operations other than the particular, sequential order disclosed. For example, operations described sequentially may in some cases be rearranged or performed concurrently. Further, descriptions and disclosures provided in association with one particular aspect or implementation are not limited to that aspect or implementation and may be applied to any aspect or implementation disclosed. It will be understood that various changes and additional variations may be made and equivalents may be substituted for elements thereof without departing from the scope of the invention or the inventive concept thereof. Certain aspects and features of any given embodiment may be translated to other embodiments described herein. In addition, many modifications may be made to adapt a particular situation or device to the teachings of the invention without departing from the essential scope thereof. Therefore, it is intended that the invention not be limited to the particular implementations disclosed herein, but that the invention will include all implementations falling within the scope of the appended claims.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing aspects. The invention extends to any novel one, or any novel combination, of the features disclosed in this specification (including any accompanying claims, abstract, and drawings), or to any novel one, or any novel combination, of the steps of any method or process so disclosed.

Throughout this application, various publications and patent applications are referenced. The disclosures of these publications in their entireties are hereby incorporated by reference into this application in order to more fully describe the state of the art to which this disclosure pertains. However, it should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

“Optional” or “optionally” means that the subsequently described event or circumstance may or may not occur, and that the description includes instances where said event or circumstance occurs and instances where it does not.

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

The terms “proximal” and “distal” as used herein refer to regions of the medical catheter system or disinfection insert. “Proximal” means a region closer to the light source (and closer to the practitioner during a procedure), whereas “distal” means a region farther from the light source (and farther from the practitioner during a procedure).

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

As used herein, disinfection means killing a pathogen, immobilizing the pathogen, reducing numbers of the pathogen, neutralizing the pathogen, or otherwise reducing the virulence of the pathogen. “Pathogen” can indicate any form of bacteria, virus, fungus, or parasite.

### REFERENCES

Agocs, M.; Clarkson, T., Mercury Toxicity. CDC Case Study. 1992.
Anaissie, E.; Samonis, G.; Kontoyiannis, D.; Costerton, J.; Sabharwal, U.; Bodey, G.;
Raad, I., Role of catheter colonization and infrequent hematogenous seeding in catheter-related infections. European Journal of Clinical Microbiology and Infectious Diseases 1995, 14 (2), 134-137.
Bak, J.; Ladefoged, S. D.; Begovic, T.; Winding, A., UVC fluencies for preventative treatment of Pseudomonas aeruginosa contaminated polymer tubes. Biofouling 2010, 26 (7), 821-828.
Barraud, N.; Schleheck, D.; Klebensberger, J.; Webb, J. S.; Hassett, D. J.; Rice, S. A.; Kjelleberg, S., Nitric Oxide Signaling in Pseudomonas Aeruginosa Biofilms Mediates Phosphodiesterase Activity, Decreased Cyclic di-GMP levels, and Enhanced Dispersal. J. Bacteriol. 2009, 191 (23), 7333-7342.
Brisbois, E. J.; Davis, R. P.; Jones, A. M.; Major, T. C.; Bartlett, R. H.; Meyerhoff, M. E.; Handa, H., Reduction in Thrombosis and Bacterial Adhesion with 7 Day Implantation of S-nitroso-N-acetylpenicillamine (SNAP)-Doped Elast-eon E2As Catheters in Sheep. J. Mater. Chem. B 2015, 3 (8), 1639-1645.
Brisbois, E. J.; Handa, H.; Major, T. C.; Bartlett, R. H.; Meyerhoff, M. E., Long-term Nitric Oxide Release and Elevated Temperature Stability with S-nitroso-N-acetylpenicillamine (SNAP)-doped Elast-eon E2As Polymer. Biomaterials 2013, 34 (28), 6957-6966.
Brisbois, E. J.; Kim, M.; Wang, X.; Mohammed, A.; Major, T. C.; Wu, J.; Brownstein, J.; Xi, C.; Handa, H.; Bartlett, R. H.; Meyerhoff, M. E., Improved Hemocompatibility of Multilumen Catheters via Nitric Oxide (NO) Release from S-nitroso-N-acetylpenicillamine (SNAP) Composite Filled Lumen. ACS Appl. Mater. Interfaces 2016, 8 (43), 29270-29279.
Brisbois, E. J.; Major, T. C.; Goudie, M. J.; Bartlett, R. H.; Meyerhoff, M. E.; Handa, H., Improved Hemocompatibility of Silicone Rubber Extracorporeal Tubing via Solvent Swelling-impregnation of S-nitroso-N-acetylpenicillamine (SNAP) and Evaluation in Rabbit Thrombogenicity Model. Acta Biomater. 2016, 37, 111-119.
Brisbois, E. J.; Major, T. C.; Goudie, M. J.; Meyerhoff, M. E.; Bartlett, R. H.; Handa, H., Attenuation of Thrombosis and Bacterial Infection Using Dual Function Nitric Oxide Releasing Central Venous Catheters in a 9 Day Rabbit Model. Acta Biomater. 2016, 44, 304-312.
Bryan, N. S., Nitric oxide enhancement strategies. Future science office action 2015, 1 (1).
Bryers, J. D., Medical biofilms. Biotechnology and bioengineering 2008, 100 (1), 1-18. Chipinda, I.; Simoyi, R. H., Formation and Stability of a Nitric Oxide Donor: S-nitroso-N-acetylpenicillamine. J. Phys. Chem. B 2006, 110 (10), 5052-5061.
Chug, M. K.; Feit, C.; Brisbois, E. J., Increasing the Lifetime of Insulin Cannula with Antifouling and Nitric Oxide Releasing Properties. ACS Applied Bio Materials 2019.
Colletta, A.; Wu, J.; Wo, Y.; Kappler, M.; Chen, H.; Xi, C.; Meyerhoff, M. E., S-Nitroso-N-acetylpenicillamine (SNAP) Impregnated Silicone Foley Catheters: A Potential Biomaterial/Device to Prevent Catheter-Associated Urinary Tract Infections. ACS Biomater. Sci. Eng. 2015, 1 (6), 416-424.
Coneski, P. N.; Schoenfisch, M. H., Nitric Oxide Release: Part Iii. Measurement and Reporting. Chemical Society Reviews, 2012, 41 (10), 3753-3758.
Cosgrove, S. E.; Qi, Y.; Kaye, K. S.; Harbarth, S.; Karchmer, A. W.; Carmeli, Y., The impact of methicillin resistance in Staphylococcus aureus bacteremia on patient outcomes: mortality, length of stay, and hospital charges. Infect. Control Hosp. Epidemiol. 2005, 26 (2), 166-174.
Dai, T.; Gupta, A.; Murray, C. K.; Vrahas, M. S.; Tegos, G. P.; Hamblin, M. R., Blue light for infectious diseases: Propionibacterium acnes, Helicobacter pylori, and beyond? Drug Resistance Updates 2012, 15 (4), 223-236.
Douglass, M. E.; Goudie, M. J.; Pant, J.; Singha, P.; Hopkins, S.; Devine, R.; Schmiedt, C. W.; Handa, H., Catalyzed Nitric Oxide Release via Cu Nanoparticles Leads to an Increase in Antimicrobial Effects and Hemocompatibility for Short-Term Extracorporeal Circulation. ACS Applied Bio Materials 2019.
Eaglstein, W. H.; Marsico, A. R., Dichotomy in response to indomethacin in UV-C and UV-B induced ultraviolet light inflammation. Journal of Investigative Dermatology 1975, 65 (2), 238-240.
Enwemeka, C. S.; Williams, D.; Hollosi, S.; Yens, D.; Enwemeka, S. K., Visible 405 nm SLD light photo-destroys methicillin-resistant Staphylococcus aureus (MRSA) in vitro. Lasers in Surgery and Medicine: The Official Journal of the American Society for Laser Medicine and Surgery 2008, 40 (10), 734-737.
Fang, F. C., Antimicrobial actions of reactive oxygen species. MBio 2011, 2 (5).
Fang, F. C., Antimicrobial reactive oxygen and nitrogen species: concepts and controversies. Nature Reviews Microbiology 2004, 2 (10), 820.
Fang, F. C., Perspectives series: host/pathogen interactions. Mechanisms of nitric oxide-related antimicrobial activity. The Journal of clinical investigation 1997, 99 (12), 2818-2825.
Feit, C.; Chug, M. K.; Brisbois, E. J., Development of S-Nitroso-N-Acetylpenicillamine (SNAP) Impregnated Medical Grade Polyvinyl Chloride for Antimicrobial Medical Device Interfaces. ACS Applied Bio Materials 2019.
Frost, M. C.; Meyerhoff, M. E., Controlled Photoinitiated Release of Nitric Oxide from Polymer Films Containing S-nitroso-N-acetyl-DL-penicillamine Derivatized Fumed Silica Filler. Journal of the American Chemical Society 2004, 126 (5), 1348-1349.
Gad, F.; Zahra, T.; Hasan, T.; Hamblin, M. R., Effects of growth phase and extracellular slime on photodynamic inactivation of gram-positive pathogenic bacteria. Antimicrobial agents and chemotherapy 2004, 48 (6), 2173-2178.
Gates, F. L., A study of the bactericidal action of ultra violet light: III. The absorption of ultra violet light by bacteria. The Journal of general physiology 1930, 14 (1), 31-42.
Gierke, G. E.; Nielsen, M.; Frost, M. C., S-Nitroso-N-acetyl-D-penicillamine Covalently Linked to Polydimethylsiloxane (SNAP-PDMS) for Use as a Controlled Photoinitiated Nitric Oxide Release Polymer. Sci. Technol. Adv. Mater. 2011, 12 (5), 055007.
Goudie, M. J.; Brisbois, E. J.; Pant, J.; Thompson, A.; Potkay, J. A.; Handa, H., Characterization of an S-nitroso-N-acetylpenicillamine-Based Nitric Oxide Releasing Polymer from a Translational Perspective. Int. J. Polym. Mater. Polym. Biomater. 2016, 65 (15), 769-778.
Goudie, M. J.; Pant, J.; Handa, H., Liquid-infused nitric oxide-releasing (LINORel) silicone for decreased fouling, thrombosis, and infection of medical devices. Sci. Rep. 2017, 7 (1), 13623.
Handa, H.; Major, T. C.; Brisbois, E. J.; Amoako, K. A.; Meyerhoff, M. E.; Bartlett, R. H., Hemocompatibility Comparison of Biomedical Grade Polymers Using Rabbit Thrombogenicity Model for Preparing Nonthrombogenic Nitric Oxide Releasing Surfaces. J. Mater. Chem. B 2014, 2 (8), 1059-1067.
Hetrick, E. M.; Schoenfisch, M. H., Analytical Chemistry of Nitric Oxide. In Annual Review of Analytical Chemistry, 2009, pp 409-433.
Hibbs Jr, J.; Taintor, R.; Vavrin, Z., Science 1987, 235, 473-476. There is no corresponding record for this reference.[Google Scholar].
Hopkins, S. P.; Pant, J.; Goudie, M. J.; Schmiedt, C.; Handa, H., Achieving Long-term Biocompatible Silicone via Covalently Immobilized S-nitroso-N-acetylpenicillamine (SNAP) that Exhibits 4 Months of Sustained Nitric Oxide Release. ACS Appl. Mater. Interfaces 2018.
Hopkins, S. P.; Pant, J.; Goudie, M. J.; Schmiedt, C.; Handa, H., Achieving Long-Term Biocompatible Silicone Via Covalently Immobilized S-Nitroso-N-Acetylpenicillamine (Snap) That Exhibits 4 Months of Sustained Nitric Oxide Release. ACS Appl. Mater. Interfaces 2018.
Hu, X.; Huang, Y.-Y.; Wang, Y.; Wang, X.; Hamblin, M. R., Antimicrobial photodynamic therapy to control clinically relevant biofilm infections. Front Microbiol. 2018, 9, 1299.
Jones, M.; Weaver, A.; Weller, W., The relative effectiveness of some chelating agents as antidotes in acute cadmium poisoning. Research communications in chemical pathology and pharmacology 1978, 22 (3), 581-588.
Kark, R. A. P.; Poskanzer, D. C.; Bullock, J. D.; Boylen, G., Mercury Poisoning and Its Treatment with N-Acetyl-D,L-Penicillamine. New England Journal of Medicine 1971, 285 (1), 10-16.
Kim YS, S. M., Park J D, et al., Subchronic oral toxicity of silver nanoparticles. Particle and Fibre Toxicology 2010, (7), 20.
Klevens, R. M.; Jonathan, R. E.; Chesley, L. R., Jr.; Teresa, C. H.; Robert, P. G.; Daniel, A. P.; Denise, M. C., Estimating Health Care-Associated Infections and Deaths in U.S. Hospitals, 2002. Public Health Rep. (122) 2007, (2), 160-166.
Klubben, W. S.; Logunov, S. L.; Fewkes, E. J.; Mooney, J.; Then, P. M.; Wigley, P. G.; Schreiber, H.; Matias, K.; Wilson, C. J.; Ocampo, M. Novel light diffusing fiber for use in medical applications, Optical Fibers and Sensors for Medical Diagnostics and Treatment Applications XVI, International Society for Optics and Photonics: 2016; p 970218.
Lautner, G.; Meyerhoff, M. E.; Schwendeman, S. P., Biodegradable Poly(lactic-co-glycolic acid) Microspheres Loaded with S-nitroso-N-acetyl-D-penicillamine for Controlled Nitric Oxide Delivery. J. Controlled Release 2016, 225, 133-139.
Lautner, G.; Stringer, B.; Brisbois, E. J.; Meyerhoff, M. E.; Schwendeman, S. P., Controlled Light-Induced Gas Phase Nitric Oxide Release from S-nitrosothiol-doped Silicone Rubber Films. Nitric Oxide 2019.
Lipovsky, A.; Nitzan, Y.; Gedanken, A.; Lubart, R., Visible light-induced killing of bacteria as a function of wavelength: Implication for wound healing. Lasers in surgery and medicine 2010, 42 (6), 467-472.
Maki, D. G.; Kluger, D. M.; Crnich, C. J. In The risk of bloodstream infection in adults with different intravascular devices: a systematic review of 200 published prospective studies, Mayo Clinic Proceedings, Elsevier: 2006; pp 1159-1171.
Marazzi, M.; López-Delgado, A.; Fernández-González, M. A.; Castano, O.; Frutos, L. M.; Temprado, M., Modulating Nitric Oxide Release by S-Nitrosothiol Photocleavage: Mechanism and Substituent Effects. The Journal of Physical Chemistry A 2012, 116 (26), 7039-7049.
Mase, J. D.; Razgoniaev, A. O.; Tschirhart, M. K.; Ostrowski, A. D., Light-controlled release of nitric oxide from solid polymer composite materials using visible and near infra-red light. Photochemical & Photobiological Sciences 2015, 14 (4), 775-785.
Massoumi, M., Kumar, R., Chug, M. K., Qian, Y., Brisbois, E. Nitric Oxide Release and Antibacterial Efficacy Analyses of S-Nitroso-N-Acetyl-Penicillamine Conjugated to Titanium Dioxide Nanoparticles. ACS Appl. Bio Mater. 2022, 5 (5), 2285-2295.
Mendez-Pfeiffer, P. A.; Soto Urzna, L.; Sánchez-Mora, E.; Gonzá-lez, A. L.; Romo-Herrera, J. M.; Gervacio Arciniega, J. J.; Martinez Morales, L. J., Damage on Escherichia coli and Staphylococcus aureus using white light photoactivation of Au and Ag nanoparticles. Journal of Applied Physics 2019, 125 (21), 213102.
Mermel, L. A.; Farr, B. M.; Sherertz, R. J.; Raad, I. I.; O'Grady, N.; Harris, J. S.; Craven, D. E., Guidelines for the Management of Intravascular Catheter-Related Infections. Clinical Infectious Diseases 2001, 32 (9), 1249-1272.
O'grady, N. P.; Alexander, M.; Burns, L. A.; Dellinger, E. P.; Garland, J.; Heard, S. O.; Lipsett, P. A.; Masur, H.; Mermel, L. A.; Pearson, M. L., Guidelines for the prevention of intravascular catheter-related infections. Clinical infectious diseases 2011, 52 (9), e162-e193.
Osma, S.; Kahveci, Ş.; Kaya, F.; Akalin, H.; Özakin, C.; Yilmaz, E.; Kutlay, O., Efficacy of antiseptic-impregnated catheters on catheter colonization and catheter-related bloodstream infections in patients in an intensive care unit. J. Hosp. Infect. 2006, 62 (2), 156-162.
Pant, J.; Gao, J.; Goudie, M. J.; Hopkins, S. P.; Locklin, J.; Handa, H., A Multi-Defense Strategy: Enhancing Bactericidal Activity of a Medical Grade Polymer with a Nitric Oxide Donor and Surface-immobilized Quaternary Ammonium Compound. Acta Biomater. 2017, 58, 421-431.
Pant, J.; Goudie, M. J.; Hopkins, S. P.; Brisbois, E. J.; Handa, H., Tunable Nitric Oxide Release from S-nitroso-N-acetylpenicillamine via Catalytic Copper Nanoparticles for Biomedical Applications. ACS Appl. Mater. Interfaces 2017, 9 (18), 15254-15264.
Politano, A. D.; Campbell, K. T.; Rosenberger, L. H.; Sawyer, R. G., Use of silver in the prevention and treatment of infections: silver review. Surgical infections 2013, 14 (1), 8-20. Rasmussen, T. B.; Givskov, M., Quorum-sensing inhibitors as anti-pathogenic drugs. International Journal of Medical Microbiology 2006, 296 (2-3), 149-161.
Reynolds, M. M.; Frost, M. C.; Meyerhoff, M. E., Nitric Oxide-Releasing Hydrophobic Polymers: Preparation, Characterization, and Potential Biomedical Applications. Free Radical Biol. Med. 2004, 37 (7), 926-936.
Schairer, D. O.; Chouake, J. S.; Nosanchuk, J. D.; Friedman, A. J., The Potential of Nitric Oxide Releasing Therapies as Antimicrobial Agents. Virulence 2012, 3 (3), 271-279.
Singha, P.; Pant, J.; Goudie, M. J.; Workman, C. D.; Handa, H., Enhanced Antibacterial Efficacy of Nitric Oxide Releasing Thermoplastic Polyurethanes with Antifouling Hydrophilic Topcoats. Biomater. Sci. 2017, 5 (7), 1246-1255.
Vaughn, M. W.; Kuo, L.; Liao, J. C., Estimation of Nitric Oxide Production and Reactionrates in Tissue by Use of a Mathematical Model. Am. J. Physiol. Heart Circ. Physiol. 1998, 274 (6), H2163-H2176.
Veenstra, D. L.; Saint, S.; Saha, S.; Lumley, T.; Sullivan, S. D., Efficacy of antiseptic-impregnated central venous catheters in preventing catheter-related bloodstream infection: a meta-analysis. Jama 1999, 281 (3), 261-267.
Williams, D. L. H., The Chemistry of S-nitrosothiols. Acc. Chem. Res. 1999, 32 (10), 869-876.
Wink, D. A.; Mitchell, J. B., Chemical biology of nitric oxide: insights into regulatory, cytotoxic, and cytoprotective mechanisms of nitric oxide. Free Radical Biol. Med. 1998, 25 (4-5), 434-456.
Wo, Y.; Brisbois, E. J.; Wu, J.; Li, Z.; Major, T. C.; Mohammed, A.; Wang, X.; Colletta, A.; Bull, J. L.; Matzger, A. J.; Xi, C.; Bartlett, R. H.; Meyerhoff, M. E., Reduction of Thrombosis and Bacterial Infection via Controlled Nitric Oxide (NO) Release from S-nitroso-N-acetylpenicillamine (SNAP) Impregnated CarboSil Intravascular Catheters. ACS Biomater. Sci. Eng. 2017, 3 (3), 349-359.
Wo, Y.; Li, Z.; Brisbois, E. J.; Colletta, A.; Wu, J.; Major, T. C.; Xi, C.; Bartlett, R. H.; Matzger, A. J.; Meyerhoff, M. E., Origin of Long-Term Storage Stability and Nitric Oxide Release Behavior of CarboSil Polymer Doped with S-nitroso-N-acetyl-D-penicillamine. ACS Appl. Mater. Interfaces 2015, 7 (40), 22218-22227.
Wo, Y.; Li, Z.; Colletta, A.; Wu, J.; Xi, C.; Matzger, A. J.; Brisbois, E. J.; Bartlett, R. H.; Meyerhoff, M. E., Study of Crystal Formation and Nitric Oxide (NO) Release Mechanism from S-nitroso-N-acetylpenicillamine (SNAP)-doped CarboSil Polymer Composites for Potential Antimicrobial Applications. Composites, Part B 2017, 121, 23-33.
Wood, P. D.; Mutus, B.; Redmond, R. W., The mechanism of photochemical release of nitric oxide from S-nitrosoglutathione. Photochem. Photobiol. 1996, 64 (3), 518-524.
Zhu, X.; Oh, H.-S.; Ng, Y. C. B.; Tang, P. Y. P.; Barraud, N.; Rice, S. A., Nitric oxide-mediated induction of dispersal in Pseudomonas aeruginosa biofilms is inhibited by flavohemoglobin production and is enhanced by imidazole. Antimicrobial agents and chemotherapy 2018, 62 (3).

1. A disinfection system for medical tubing, the disinfection system comprising:
a disinfection insert configured to extend within a lumen of a medical tubing, the disinfection insert comprising:
a fiber optic; and
a polymer surrounding at least a portion of the fiber optic, the polymer comprising a nitric oxide (NO) donor molecule, wherein the NO donor molecule is releasable upon illumination of the polymer by the fiber optic.

2. The disinfection system of claim 1, wherein the NO donor molecule is an S-nitrosothiol.

3. The disinfection system claim 1, wherein the NO donor molecule is releasable at a flux between 0.1×10⁻¹⁰ mol cm⁻² min⁻¹ and 100×10⁻¹⁰ mol cm⁻² min⁻¹.

4. The disinfection system of claim 1, wherein the fiber optic is a side glow fiber optic.

5. The disinfection system of claim 1, wherein the polymer is coated directly onto the fiber optic.

6. (canceled)

7. The disinfection system of claim 1, wherein the polymer is one of silicone rubber, siloxane-based polyurethane elastomer, or thermoplastic silicone-polycarbonate urethane.

8. (canceled)

9. (canceled)

10. The disinfection system of claim 1, wherein the disinfection insert comprises a fastener configured to removably attach the disinfection insert to the medical tubing.

11. (canceled)

12. (canceled)

13. The disinfection system of claim 1, further comprising a light source in optical communication with the fiber optic and configured to selectively illuminate the fiber optic.

14. (canceled)

15. The disinfection system of claim 13, wherein the light source is removably attached to the disinfection insert.

16. The disinfection system of claim 13, wherein the light source delivers light of wavelengths ranging from 200 nanometers to 700 nanometers.

17. (canceled)

18. (canceled)

19. The disinfection system of claim 13, wherein a light source controller controls the wavelength of light from the light source, an intensity of light from the light source, or both a wavelength and intensity of light from the light source.

20. (canceled)

21. (canceled)

22. A method of making a disinfection insert for medical tubing, the method comprising:
incorporating a nitric oxide (NO) donor molecule into a polymer; and
coupling a fiber optic to the polymer, wherein illumination of the fiber optic while the disinfection insert is inserted into the medical tubing causes the NO donor molecule to be released by the polymer.

23. The method of claim 22, wherein coupling the fiber optic to the polymer comprises dipping a portion of the fiber optic into a liquid form of the polymer, such that the polymer coats at least a portion of the fiber optic.

24. The method of claim 22, wherein coupling the fiber optic to the polymer comprises attaching a solid form of the polymer to the fiber optic such that the polymer surrounds at least a portion of the fiber optic.

25. (canceled)

26. (canceled)

27. (canceled)

28. (canceled)

29. (canceled)

30. (canceled)

31. The method of claim 22, further comprising coupling the fiber optic to a light source, wherein the light source is further coupled to a light source controller for selectively activating the light source to illuminate the fiber optic.

32. A method of disinfecting medical tubing using the disinfection system of claim 1, the method comprising:
providing the disinfection insert, wherein the disinfection insert is inserted into a lumen of the medical tubing; and
activating a light source to illuminate the disinfection insert, thereby causing the release of the NO donor molecule from the polymer of the disinfection insert, wherein the NO donor molecule contacts pathogens on or within the medical tubing upon being released, thereby inactivating at least a portion of the pathogens on or within the tubing via contact with the NO donor molecule.

33. (canceled)

34. (canceled)

35. (canceled)

36. (canceled)

37. (canceled)

38. The method of claim 32, wherein the medical tubing is a medical catheter.

39. (canceled)

40. The method of claim 32, wherein the medical tubing is one of an endotracheal tube, a wound dressing or wound patch, a photodynamic therapy device, a cardiopulmonary bypass device, a hemodialysis device, a medical port, a feeding tube, or an intestinal tube.

41. (canceled)

42. (canceled)

43. The method of claim 32, further comprising controlling the light source to modulate at least one of an intensity of light from the light source or a wavelength of the light from the light source to vary a flux of the NO donor molecule.

44. (canceled)

45. The method of claim 32, wherein activating the light source comprises transmitting a control signal from a light source controller to the light source.
